(19) United States
(12) Patent Application Publication
(10) Pub. No.: US 2021/0302416 A1
(43) Pub. Date: Sep. 30, 2021

(54) METHOD FOR DETECTING BIOMARKERS

(71) Applicant: Veravas, Inc., Charleston, SC (US)

(72) Inventors: Joshua Caine Soldo, Prior Lake, MN (US); Scott Douglas Bergmann, River Falls, WI (US); Carmen Leah Wiley, Spokane, WI (US)

(21) Appl. No.: 17/263,809

(22) PCT Filed: Jul. 26, 2019
(86) PCT No.: PCT/US19/43715

§ 371 Date: Jan. 27, 2021

Related U.S. Application Data
(60) Provisional application No. 62/711,415, filed on Jul. 27, 2018.
(60) Provisional application No. 62/747,017, filed on Oct. 17, 2018.

(51) Int. Cl. G01N 33/543; G01N 33/74

(52) U.S. Cl. CPC G01N 33/54326; G01N 2800/2864; G01N 33/74

(57) ABSTRACT

The present invention is directed to methods for using a plurality of particles comprising surfaces each independently comprising a capture moiety to isolate and characterize biomarkers (e.g., for obstructive sleep apnea).

### CROSS-REFERENCE TO RELATED APPLICATIONS

This application claims the benefit of priority to U.S. Provisional Patent Application No. 62/711,415 filed Jul. 27, 2018, and U.S. Provisional Patent Application No. 62/747,017 filed Oct. 17, 2018. The entire content of each of the aforementioned applications is incorporated herein by reference for all purposes.

### FIELD OF THE INVENTION

The present invention relates to methods for using a plurality of particles (e.g, microparticulate, nanoparticulate; magnetic, non-magnetic) comprising surfaces each independently comprising a capture moiety as described herein to isolate and characterize biomarkers (e.g., for obstructive sleep apnea).

### BACKGROUND OF THE INVENTION

Laboratory testing plays a critical role in health assessment, health care, and ultimately the public's health, and affects persons in every life stage. Almost everyone will experience having one or more laboratory tests conducted during their lifetime. An estimated 7 to 10 billion laboratory tests are performed each year in the United States alone, and laboratory test results influence approximately 70% of medical decisions.

In addition, since the Centers for Medicare and Medicaid Services (CMS) on Jan. 1, 2018 implemented the new Clinical Laboratory Fee Schedule (CLFS) as required by the Protecting Access to Medicare Act (PAMA), PAMA is reducing lab testing reimbursement. It is even more critical lab results are accurate the first time and troubleshooting efforts are reduced or take less time and do not impact lab workflow.

Interference is a substance present in a patient specimen that can alter the correct value of the result of a diagnostic test, e.g., by interfering with antibody binding, or that can increase or decrease assay signal by bridging, steric hindrance, or autoantibody mechanisms. While it is known that immunoassays are susceptible to interference, the clinical laboratory may still report erroneous results if such results are not recognized and flagged by the instrument (analyser) or laboratory, or if the physician does not notify the laboratory that the patient result does not fit the clinical picture. Erroneous results can occur unexpectedly with any specimen without the practical means to identify upfront such specimens likely to cause problems. The consequence of such interference is that erroneous results can result in false negatives and false positive test results, that can impact patient care, and can lead to unnecessary invasive, diagnostic or therapeutic procedures, or failure to treat a patient.

Notwithstanding the complications arising from interference, biomarker(s) screening and diagnostic testing can be difficult, for example because of their low presence or abundance in a biological sample.

Urine and/or blood biomarkers for Obstructive Sleep Apnea (OSA) can be used to facilitate the diagnosis and cost-effective treatment of the disease in children and adults. The overall goal of urine and/or blood biomarkers is to address and measure the optimal biomarker signature for OSA. OSA is a highly prevalent disorder in children and adults associated with increased risk for cardiovascular disease, diabetes, and other chronic conditions. Unfortunately, up to 90% of individuals with OSA have not been diagnosed and are not receiving appropriate therapy to manage this disease. While much is known regarding the pathophysiology and consequences of OSA, the molecular mechanisms of OSA remain poorly defined. However, advances in proteomics-based technologies have facilitated the discovery of novel biomarkers as potential diagnostic and therapeutic targets for many diseases including OSA.

Epidemiological studies have indicated that OSA affects 6-13% of the adult population and 1-4% of the pediatric population. The prevalence of OSA is >50% in patients with cardiac or metabolic disorders than in the general population. Untreated OSA worsens quality of life and brings long-term consequences, which include cardiovascular disease, hypertension, diabetes, obesity, stroke, depression, and a variety of metabolic disorders. In children, OSA can lead to cognitive and behavioral disorders, and can be misdiagnosed as attention deficit disorder (ADD).

Currently, the definitive diagnosis of OSA requires an overnight sleep study with laboratory-based polysomnography (PSG). However, sleep studies are laborious, expensive, inaccessible, and inconvenient for diagnosing OSA in children and adults. While home-based unattended studies using a portable sleep monitor has been encouraged, 56% of home-based diagnosed OSA still require confirmation by a laboratory PSG sleep study. There is a need for early identification of OSA in patients by a simple and inexpensive screening or diagnostic test to prevent OSA consequences such as a technology that 1) simplifies the discovery and analysis of potential OSA biomarkers in known OSA positive patient samples, and 2) combines multiple biomarkers in a panel to achieve the desired sensitivity and specificity for an accurate diagnosis of OSA and to access efficacy of OSA treatment.

There is a need to develop obstructive sleep apnea (OSA) diagnostic testing solutions where the accurate, precise and sensitive measurement of low abundance biomarkers is key to correctly rule-in/rule-out OSA and improve patient outcomes. 43 million Americans may have OSA which can lead to obesity, hypertension, heart disease, diabetes, behavioral issues and learning problems, and can be misdiagnosed as attention deficit hyperactivity disorder (ADHD) in children. The CDC estimates the prevalence of pediatric OSA in the US at around 1 million. OSA is one of the most under diagnosed conditions today and OSA diagnosis conventionally requires an expensive and inconvenient sleep study (polysomnography). In the pediatric population parents are often unwilling and children can be uncooperative to endure a sleep study.

There is therefore a clinical need for simple, inexpensive, automatable and effective solutions to eliminate or minimize sample interference and enrich biomarker(s) concentration prior to diagnostic testing without impacting laboratory workflow and turnaround time.

### SUMMARY OF THE INVENTION

Described herein are methods for the simple, efficient and cost-effective detection of one or more biomarkers in biological samples to, e.g., manage and mitigate a multitude of known sample-specific interferences that can lead to erroneous test results and increased risk to patient safety, such as heterophilic antibodies in patients who have been treated with monoclonal mouse antibodies or have received them for diagnostic purposes. The methods described herein can also manage and mitigate sample-specific interferences that arise from biotin that can come from over the counter (OTC) supplements, multivitamins and herbal remedies taken by consumers for health & beauty and weight loss or therapeutically, e.g., for the treatment of multiple sclerosis.

In an aspect, provided is a method for removing biomarkers from a biological sample, the method comprising: a) combining the sample with a plurality of particles, wherein each particle independently comprises a capture moiety (i.e., a type or species of capture moiety), to provide a mixture; b) mixing the mixture to provide one or more particle complexes to the biomarkers; and c) removing or isolating the particle complexes to provide an depleted solution; thereby removing biomarkers from the biological sample.

In an aspect, provided is a method for isolating biomarkers from a biological sample, the method comprising: a) combining the sample with a plurality of particles, wherein each particle independently comprises a capture moiety (i.e., a type or species of capture moiety), to provide a mixture; b) mixing the mixture to provide one or more particle complexes to the biomarkers; and c) removing or isolating the particle complexes to provide an depleted solution and an enriched isolate; thereby isolating biomarkers from the biological sample.

### DETAILED DESCRIPTION OF THE INVENTION

Described herein are methods for isolating or enriching one or more biomarkers for obstructive sleep apnea (OSA) in a biological sample, the method comprising combining a plurality of particles as described herein with a biological sample as described herein.

In an aspect, described herein is a method for removing biomarkers for OSA from a biological sample, the method comprising: a) combining the sample with a plurality of particles, wherein each particle independently comprises a capture moiety (i.e., a type or species of capture moiety), to provide a mixture; b) mixing the mixture to provide one or more particle complexes to the biomarkers; and c) removing or isolating the particle complexes to provide a depleted solution; thereby removing biomarkers from the biological sample.

In an aspect, described herein is a method for isolating biomarkers from a biological sample, the method comprising: a) combining the sample with a plurality of particles, wherein each particle independently comprises a capture moiety (i.e., a type or species of capture moiety), to provide a mixture; b) mixing the mixture to provide one or more particle complexes to the biomarkers; and c) removing or isolating the particle complexes to provide a depleted solution and an enriched isolate; thereby isolating biomarkers from the biological sample. In some embodiments, the plurality of particles comprises a plurality of capture moieties (e.g., a plurality of particles each independently covalently or non-covalently bonded to a plurality of capture moieties).

In some embodiments, a conditioning agent is added to the biological sample prior to combining the sample with the plurality of particles. In some embodiments, the conditioning agent is a pH adjustment agent, a molarity adjustment agent, an interference blocking agent, or a liberation or release agent.

In some embodiments, the plurality of particles comprises a first particle comprising a first capture moiety. In some embodiments, the plurality of particles comprises a second particle comprising a second capture moiety. In some embodiments, the plurality of particles comprises a third particle comprising a third capture moiety. In some embodiments, the plurality of particles comprises a fourth particle comprising a fourth capture moiety. In some embodiments, the plurality of particles comprises a fifth particle comprising a fifth capture moiety. In some embodiments, the plurality of particles comprises a sixth particle comprising a sixth capture moiety. In some embodiments, the plurality of particles comprises a seventh particle comprising a seventh capture moiety. In some embodiments, the plurality of particles comprises an eighth particle comprising an eighth capture moiety. In some embodiments, the plurality of particles comprises a ninth particle comprising a ninth capture moiety. In some embodiments, the plurality of particles comprises a tenth particle comprising a tenth capture moiety. In some embodiments, the method comprises removing or isolating a first, second, third, fourth, fifth, sixth, seventh, eighth, ninth, or tenth biomarker from the biological sample.

In some embodiments, the method further comprises adding to the mixture a cleavage reagent or releasing agent to provide an enriched isolate.

In some embodiments, the method further comprises performing a diagnostic test on the biomarker(s) (e.g., after the method of removing or method of isolating described herein). In some embodiments, the diagnostic test detects the presence or absence of two or more biomarkers concurrently.

In some embodiments, the first particle is different in size, shape, chemistry, color, or other characteristic than the second particle. In some embodiments, the first particle is different in size, shape, chemistry, color, or other characteristic than the third particle. In some embodiments, the first particle is different in size, shape, chemistry, color, or other characteristic than the fourth particle. In some embodiments, the first particle is different in size, shape, chemistry, color, or other characteristic than the fifth particle. In some embodiments, the first particle is different in size, shape, chemistry, color, or other characteristic than the sixth particle. In some embodiments, the first particle is different in size, shape, chemistry, color, or other characteristic than the seventh particle. In some embodiments, the first particle is different in size, shape, chemistry, color, or other characteristic than the eighth particle. In some embodiments, the first particle is different in size, shape, chemistry, color, or other characteristic than the ninth particle. In some embodiments, the first particle is different in size, shape, chemistry, color, or other characteristic than the tenth particle. In some embodiments, the characteristic is selectivity, affinity, or avidity to a biomarker(s) described herein.

In some embodiments, the size is 50-1000 nm in diameter, for example, 50-500 nm in diameter, 50-300 nm in diameter, 50 nm to 100 nm in diameter, 200 nm to 600 nm in diameter, 400 nm to 600 nm in diameter, or 100 nm to 500 nm in diameter. In some embodiments, the size is 1-3 micron in diameter. In some embodiments, the particle is 5 nm to 100 nm in diameter.

In some embodiments, a first population of particles is present at a greater concentration than a second population of particles. In some embodiments, a first population of particles is present at a greater concentration than a third population of particles. In some embodiments, a first population of particles is present at a greater concentration than a fourth population of particles. In some embodiments, the ratio of a first particle to a second particle is 1:1, 1:2, 1:3, 1:4, 1:5, 1:6, 1:7, 1:8, 1:9, 1:10. Concentrations of particles are provided in units of mass per unit volume (e.g., mg/mL, g/L) or % solids (w/v). For example, 0.50% w/v is equivalent to 5 mg/mL or 5 g/L.

In some embodiments, the first particle is present at a first concentration. In some embodiments, the second particle is present at a second concentration. In some embodiments, the third particle is present at a third concentration. In some embodiments, the fourth particle is present at a fourth concentration. In some embodiments, the fifth particle is present at a fifth concentration. In some embodiments, the sixth particle is present at a sixth concentration. In some embodiments, the seventh particle is present at a seventh concentration. In some embodiments, the eighth particle is present at an eighth concentration. In some embodiments, the ninth particle is present at a ninth concentration. In some embodiments, the tenth particle is present at a tenth concentration.

In some embodiments, the first particle is a control particle (e.g., particle comprising a label or indicator (e.g., a label or indicator of known quantity, abundance). In some embodiments, the label or indicator provides measurement of concentration or volume of the sample. In some embodiments, the label or indicator provides indication of lot number or batch number of the sample. In some embodiments, the label or indicator provides a measurement of yield or particle recovery.

In some embodiments, the biomarker is biotin, HAMA, RF, Heterophilic, or anti-SAv. In some embodiments, the biomarker is an indicator of bacterial infection. In some embodiments, the biomarker is a capture moiety for a bacterium.

In some embodiments, removing or isolating the particle complexes comprises cleaving, eluting, or selectively releasing a capture moiety-biomarker complex. In some embodiments, the capture moiety (e.g., the capture moiety of a capture moiety-biomarker complex) comprises a signal detection molecule (or is pre-labeled with a signal detection molecule) for measurement in a test system. Suitable signal detection molecules include, but are not limited to, HRP, ALP, acribinium ester, isoluminol/luminol, ruthenium, N-(4-aminobutyl)-N-ethylisoluminol (ABEI)/cyclic ABEI, or fluorescein.

In some embodiments, prior to the combining step a) in the methods disclosed herein for removing or isolating biomarkers, the sample is pre-treated to remove or deplete an interference. In some embodiments, the removal or depletion of an interference comprises: (i) combining the sample with a particle comprising a capture moiety lacking specificity for the biomarkers to provide a mixture; (ii) mixing the mixture to provide particle complexes to the interference; and (iii) removing or eliminating the particle complexes to provide a depleted solution. In some embodiments, the removing or eliminating of step (iii) is carried out magnetically, physically, or chemically.

In some embodiments, the capture moiety is a human anti-animal antibody (e.g., mouse IgG, sheep IgG, goat IgG, rabbit IgG, cow IgG, pig IgG, horse IgG). In some embodiments, the capture moiety is a heterophilic antibody (e.g., FR (Fc-specific), Fab, F(ab)′2, polymerized IgG (type 1, 2a, 2b IgG and IgG fragments, serum components). In some embodiments, the capture moiety is an assay specific binder (e.g., biotin, fluorescein, anti-fluorescein poly/Mab, anti-biotin poly/Mab, streptavidin, neutravidin). In some embodiments, the capture moiety is an assay specific signal molecule (e.g., HRP, ALP, acridinium ester, isoluminol/luminol, ruthenium, ABEI/cyclic ABEI). In some embodiments, the capture moiety is an assay specific blocker (e.g., BSA, fish skin gelatin, casein, ovalbumin, PVP, PVA). In some embodiments, the capture moiety is an assay specific conjugate linker (e.g., LC, LC-LC, PEO4, PEO16). In some embodiments, the capture moiety is an antigen autoantibody (e.g., free T3, free T4). In some embodiments, the capture moiety is a protein autoantibody (e.g., MTSH, TnI, TnT, non-cardiac TnT (skeletal muscle disease)). In some embodiments, the capture moiety is a chemiluminescent substrate (e.g., luminol, isoluminol, isoluminol derivatives, ABEI, ABEI derivatives, ruthenium, acridinium ester) or fluorescent label (e.g., fluorescein or other fluorophores and dyes). In some embodiments, the capture moiety is streptavidin, neutravidin, avidin, polyA, polyDT, aptamers, antibodies, Fab, F(ab′)2, antibody fragments, recombinant proteins, enzymes, proteins, biomolecules, polymers, or molecularly imprinted polymers. In some embodiments, the capture moiety is biotin, fluorescein, PolyDT, PolyA, antigen, etc.

In an aspect, described herein is a kit comprising a plurality of particles and an instruction booklet. In some embodiments, the kit comprises a plurality of particles, collection tube and an instruction booklet. In some embodiments, the kit comprises a plurality of particles, magnet, collection tube and an instruction booklet.

Multiplexed Application of Methods

Described herein are multiplexed applications of the methods described herein. For example, in one embodiment, more than one particle (e.g., a particle as described herein) is used to bind or complex one or more biomarkers in a biological sample. In some embodiments, a first particle comprises a first capture moiety and a second particle comprises a second moiety, wherein the first capture moiety binds or removes a first biomarker (e.g., a biomarker as described herein) and the second capture moiety binds or removes a second biomarker (e.g., a biomarker described herein), wherein when the first particle and second particle are combined with a biological sample (e.g., a biological sample as described herein), the first biomarker and second biomarker is bound or removed from the biological sample.

In some embodiments, the first particle has different physical characteristics (e.g., size, color) than the second particle.

For example, a kit for multiplexed application of the methods described herein comprise 1) blue beads coated with anti-human IgG; 2) red beads coated with anti-human IgM; 3) black beads coated with biotin-PEG. The detection reagent is a mixture of these 3 different beads for qualitative visual detection. The beads can also be labeled with unique fluorophores with different emission/excitation for multiplexed detection and semi-quantitative or quantitative results if the beads are read using a fluorimeter or fluorescence plate reader. In addition to the use of colored beads, the nanoparticles can also be labelled for analytical (fluorescent, UV/vis, chemiluminescent, electrochemiluminescent, etc.) detection.

The beads can be non-magnetic dyed beads such as latex. nanobeads (i.e. blue, red, black, green, yellow, purple, white), dyed magnetic beads (i.e. yellow, red, green), or undyed magnetic beads (brown), where the bead surface is functionalized for covalent immobilization of antibodies, proteins, antigens, antibody fragments, aptamers, oligonucleotides (PolyA, PolyDT), molecularly imprinted polymers, etc.

The microarray characterization kit comprises one or more different interference targets or analytes immobilized (i.e. covalent, affinity interaction) to each well/spot, where each well/spot will be specific for at least one mechanism of interference. When the sample, neat or diluted, is added to each well/spot the sample specific interference (only if present in the sample) interact with the immobilized interference target or analyte (e.g., biomarker, e.g., interference) only if that sample-specific interference is present. For example, if the sample comprises human anti-Goat IgG interference the human IgG will specifically bind to Goat IgG immobilized to a specific well/spot. As another example, if the sample comprises human anti-Streptavidin IgM the human IgM will specifically bind to the Streptavidin immobilized to a specific well/spot. As another example, if there is free biotin in the sample it will specifically bind to Streptavidin, neutrvidin, avidin or anti-biotin IgG immobilized to a specific well/spot. As another example, if the sample comprises 2 or more different interferences such as human anti-Sheep IgG and human anti-ruthenium IgM, when the sample is added to a well/spot comprising sheep IgG immobilized to the well/spot only the human anti-Sheep IgG will specifically bind to this well/spot while the human anti-ruthenium IgG will not bind, and when the same sample is added to a different well/spot comprising ruthenium immobilized to the well/spot only the human anti-ruthenium IgM will specifically bind to this well/spot while the human anti-sheep IgG will not bind.

After incubating each well/spot with sample, the well/spot is washed such that only human IgG, human IgM and/or biotin interference present in the sample will remain in a given well/spot only if it was specific to a interference target immobilized in a given well/spot. If there was no sample-specific interference present for a given interference target immobilized to a well/spot, then after washing there would be no detectable amount of human IgG, human IgM or biotin present in the well/spot.

For characterization of possible sample interference(s) the mixture of these 3 different beads is added to each well or reaction spot, and the blue and/or red beads will only bind to given spot if human IgG and/or human IgM has bound to the well/spot via a specific binding interaction to its interference target, and the black bead will never bind as they are only specific for biotin. The black beads will only bind to the well/reaction with immobilized Streptavidin, neutrvidin, avidin or anti-biotin IgG if the amount of free biotin in the sample is less than the biotin binding capacity or threshold for free biotin. If the free biotin in the sample exceeds the biotin binding capacity or threshold for free biotin all biotin binding sites have been occupied or saturated and none of the black beads will bind.

As for a control, there can be 3 or more wells/spots, where either human IgG, human IgM, or anti-biotin protein (Streptavidin, neutrvidin, avidin or anti-biotin IgG) is immobilized. If this mixture of 3 beads (blue, red and black) is added to a given, control well/spot, only the color bead specific for the human IgG (blue bead), human IgM. (red bed) or anti-biotin protein (black bead) will bind to the control well/spot while the other 2 colors will not bind. For example, if the control well/spot is immobilized with human IgM and the mixture of 3 color beads is added, only the red beads will bind (red beads coated with anti-human IgM) and no blue or black beads will bind. As another example, if the if the control well/spot is immobilized with anti-biotin protein and the mixture of 3 color beads is added, only the black beads will bind (black beads coated with biotin-PEG) and no blue or red beds will bind. However, if the wrong color bead binds to a control well/spot, or more than 1 color bead binds to a control well/spot, then the control FAILS. The control only PASSES if the correct color bead binds to the correct control well/spot.

However, in addition to human IgG (immunoglobin G) and IgM (immunoglobin M) there are 3 additional classes of human antibodies: IgA (immunoglobin A), IgD (immunoglobin D), and IgE (immunoglobin E). Since there are 5 classes of human antibodies, additional color beads (N=6 colors) in the detection reagent mixture can be considered to detect these possible interference mechanisms. For example, a kit can comprise: 1) blue beads coated with anti-human IgG; 2) Red beads coated with anti-human IgM; 3) Yellow beads coated with anti-human IgA; 4) Green beads coated with anti-human IgE; 5) Brown beads coated with anti-human IgD; and 6) Black beads coated with biotin-PEG.

As another example, a single color bead to detect 2 or more human antibody types such as a yellow bead co-coated with anti-human IgA, anti-human IgE and anti-human IgD, or three separate lots/batches of yellow beads, where each batch/lot is coated with a single anti-human antibody and all batches are pooled or blended into a single batch of yellow beds comprising 3 different lots of yellow beads with specificity to human IgA, IgE or IgD can be used. For example, additional color beads (N=4 colors) in the detection reagent mixture may be used to detect these possible interference mechanisms: 1) Blue beads coated with anti-human IgG; 2) Red beads coated with anti-human IgM; 3) Yellow beads coated with anti-human IgA, anti-human IgE and anti-human IgD, or a batch/pool of 3 different lots of yellow beads where one lot is coated with anti-human IgA, the second lot is coated with anti-human IgE and the third lot is coated with anti-human IgD; 4) Black beads coated with biotin-PEG.

Since IgG are bivalent (have 2 Fabs and can bind 2 moles of antigen per mole of IgG) and IgM are decavalent (have 10 Fabs and can bind 10 moles of antigen per mole IgM), it is possible that a magnetic nanoparticle depletion reagent could also be used as the characterization label in an Assay Interference Characterization Kit if the identical interference target immobilized on the magnetic nanoparticle surface is also immobilized on the microarray spot or microtiter plate well. For example, if the magnetic nanoparticle depletion reagent is coated with Sheep IgG and is added to a sample with human anti-Sheep IgG and/or human anti-Sheep IgM interference as a sample pre-treatment prior to the test to deplete human anti-sheep antibody (HASA) interference, any human anti-Sheep IgG and/or human anti-Sheep IgM will bind to the Sheep IgG immobilized on the magnetic nanoparticle surface. After this sample pre-treatment incubation is complete, the magnetic nanoparticles can be isolated from the sample using magnetic, physical or chemical separation methods and washed to remove non-bound materials from the sample. The washed magnetic nanoparticles can be reconstituted in water or buffer and added to the Assay Interference Characterization Kit wells or spots. If the well/spot also has Sheep IgG immobilized on its surface any bivalent (human IgG) and/or decavalent (human IgM) bound to the Sheep IgG on the magnetic nanoparticle surface can also capture/bind to the Sheep IgG immobilized in the well/spot to form a specific sandwich complex:
[magnetic nanoparticle]-(Sheep IgG)-{Human Anti-Sheep IgG/IgM}-(Sheep IgG)-[microarray spot or microtiter plate well).

After washing the well/spot, any brown color (i.e. magnetic nanoparticles) remaining=POSITIVE result or Human Anti-Sheep Antibody was detected in the sample. If the well/spot is clear or has no color after washing=NEGATIVE result or Human Anti-Sheep Antibody was NOT detected in the sample.

### Method of Separation

Particles described herein can be added to a collection device such as a primary blood collection tube, 24-hr urine collection device, a urine collection device, a saliva collection tube, a stool collection device, a seminal fluid collection device, a blood collection bag, or any sample collection tube or device, prior to the addition of the biological sample.

Particles described herein can also be added to a sample after collection of the sample into a collection device, or after the transfer of the sample from a primary collection device into a storage or transfer device such as a plastic or glass tube, vial, bottle, beaker, flask, bag, can, microtiter plate, ELISA plate, 96-well plate, 384-well plate 1536 well plate, cuvette, reaction module, reservoir, or any container suitable to hold, store or process a liquid sample.

In some embodiments, the particles described herein are added to a collection device comprising a biological sample. In some embodiments, the particles described herein are added to a collection device prior to addition of a biological sample.

In an aspect, described herein is a device for releasing particles comprising a collection device as described herein comprising a biological sample (i.e. screw cap which triggers release mechanism) such as on a urine collection device. For example, the device is a tube equipped with a screw cap that releases the particles described herein upon closure of the screw cap.

In an aspect, described herein is a device comprising a chemical release of particles to a container comprising a biological sample (i.e. encapsulated composition or composition that dissolves in solution at a defined rate or point in time). In some embodiments, the devices described herein are configured to delay the addition of particles described herein, for example to provide pre-treatment of sample prior to diagnostic testing.

In some embodiments, the sample described herein can be pre-treated with a chemical, protein, blocker, surfactant or combination thereof prior to addition of the particles described herein for example to adjust pH, deplete or compete for sample specific interferences, and/or manage matrix specific challenges prior to the nanoparticles being added, introduced, dispersed or mixed in the sample to improve the specificity and binding kinetics of the nanoparticles to the target biomarker(s). The delayed addition of the nanoparticles to the sample after sample pre-treatment can be controlled physically by adding the nanoparticles to the sample after sample pre-treatment. The nanoparticles can also be present in the sample during the sample pre-treatment if the nanoparticles are encapsulated, shielded or protected by a chemical, polymer or sugar shell, coating, or polymerization such that the chemical, polymer or sugar needs to dissolve before the nanoparticles can be released, added, dispersed or mixed in the sample. The delayed release of nanoparticles can use chemistry known to someone skilled in the art such as used today in delayed drug release technology.

Methods of Magnetic Separation of Particles

In one aspect, provided herein is a method for removing an interference from a biological sample (e.g., prior to a diagnostic test), or to isolate or separate magnetic particle (e.g., within a primary blood collection tube, custom sample collection device, secondary transfer tube or custom sample device). For example, a magnet-based device will quickly (less than 2 minutes; preferably less than 30 seconds) isolate the magnetic nanoparticles to the side(s) and/or bottom to form an essentially particle-free supernatant. The particle-free supernatant can be subsequently aspirated without disrupting the pellet comprising the particles and dispensed into a separate transfer tube for diagnostic testing. In some embodiments, the pellet is isolated or subjected to diagnostic testing. In some embodiments, the magnet for the magnetic separation is a multiple magnet device containing 2 to 12 magnets in a rack designed to hold 1 to 12 sample preparation tubes on a large pipetting machine. Examples of such pipetting machines include, but are not limited to, those built by Hamilton or Tecan. In some embodiments, the magnet for the magnetic separation is a multiple magnet device containing 96 or 384 magnets designed to provide magnetization to a 96 well or 384 well microtiter plate.

Methods of Physical Separation of Particles

In one aspect, described herein are methods for removing particles described herein by physical force (e.g., gravitational force). In some embodiments, the particles described herein are separated, isolated, or removed (e.g., by centrifugation) from a biological sample by physical force. In some embodiments, the methods are used prior to application of diagnostic test methods described herein, for example, within a primary blood collection tube, custom sample collection device, secondary transfer tube or custom sample device. In some embodiments, the method for removing particles is filtration.

For example, magnetic nanoparticles specific for fibronectin and/or other clotting factors or off the clot components/constituents, cellular debris (i.e. red blood cell membrane specific) for the subsequent capture or binding of the “clot” (in serum) and/or capture or binding of cellular debris (in serum or plasma) enhance centrifugation speed and efficiency (shorter spin times to improve lab efficiency, workflow and throughput) by integration of strong magnets or magnetic technology in the centrifuge rotor and/or tube holders. This combination of RCF or Gs from centrifugation with magnetic separation of the magnetic nanoparticle complex (i.e. clot+magnetic beads, cellular debris+magnetic beads) enable much quicker and more efficient separation and supernatant formation on the side or bottom of the sample tube to clarify the sample for subsequent analysis. For example, this centrifugation step in most laboratories is 4 minutes or greater, and may be reduced to 2 minutes or less (preferable 1 min or less) by combining centrifugation with magnetic separation/isolation of the magnetic nanoparticle clot/cellular debris complexes.

Moreover, if the nanoparticles or plurality of magnetic nanoparticles are also specific for one or more different sample interference mechanisms such as 1, 5, 10, 20, 30, or more different interference mechanisms, these interferences, if present, would be captured by the nanoparticles and depleted from the sample after physical separation from centrifugation, or by the combination of centrifugation and magnetic separation described above.

While these magnetic nanoparticles do not need to also have specificity to the clot or cellular debris to be isolated via centrifugation or the combination of centrifugation and magnetic separation in the centrifuge, their surface could be co-coated or immobilized with more than one antibody and/or antigen where one or more antibodies would be specific for the clot and/or cellular debris, while the other antibody(s) and/or antigen would be specific to the sample interference. In this regard, the nanoparticles would specifically bind to both sample interference as well as the clot and/or cellular debris for subsequent physical separation or isolation via centrifugation or the combination of centrifugation and magnetic separation.

The use of nanoparticles specific for the clot and/or cellular debris increase clotting rate of speed by specific binding by the magnetic nanoparticles and pulling everything to a magnetic for magnetic separation and isolation. This bead-based pellet formed by the magnetic field and strength also accelerates the clot formation based on forced proximity of the clot or specifically captured clotting factors by the nanoparticles and subsequently the magnet.

Methods of Chemical Separation of Particles

In some embodiments, the particles described herein are separated, isolated, or removed from a biological sample by chemical separation methods. In some embodiments, the chemical separation methods are used prior to application of diagnostic test methods, for example, within a primary blood collection tube, custom sample collection device, secondary transfer tube or custom sample device.

In one aspect, provided is a method for chemical separation of particles, the method comprising providing one or more of a salt, solvent, polymer, or detergent.

In some embodiments, the chemical separation methods, e.g., liquid-liquid phase separation will partition particles into a Phase A, and the nanoparticle free sample will be portioned into a Phase B where Phase B is tested. The agents for liquid-liquid phase separation (chemical phase separation) can be by salts, soluble polymers and detergents.

For example, liquid-liquid phase separation can occur by adding a non-polar solvent such as hexane to the polar aqueous sample where the particles partition into the non-polar phase leaving a nanoparticle-free aqueous phase for testing by a diagnostic test as described herein. In some embodiments, the method of separation described herein provide nanoparticles in the organic phase. In some embodiments, the method of separation described herein provide nanoparticles in the aqueous phase.

A method for isolating particles in a biological sample, the method comprising providing to the particles and biological sample a nonpolar solvent and an aqueous polar solvent to provide a nonpolar solvent layer and a polar solvent layer, removing a nonpolar solvent layer comprising the nonpolar solvent, and isolating the aqueous polar solvent comprising the particles, thereby isolating the particles.

Sample recovery can be adjusted or corrected by addition and use of an internal standard, such as a deuterated internal standard or an internal control particle for LC-MS/MS, prior to aspirating and discarding the non-polar phase.

In some embodiments, the separation is physical separation used in combination with magnetic separation. For example, in an aspect, provided is a device (e.g., a magnetized centrifuge or a centrifuge equipped with a magnet that aids in separation by both the gravity and magnetic force of a magnet). In one aspect, provided herein is a device for separation of a particle described herein, the device comprising a magnet and centrifuge. In some embodiments, the device significantly reduces the time of centrifugation.

### Method for the Removal or Enrichment of Biomarkers

Described herein are methods for enriching or increasing the concentration of a biomarker in a biological sample. “Enrichment” is defined as the complete or partial particle capture and binding of target analyte(s) or biomarker(s) to the particles from a biological sample (e.g., human or animal serum, plasma, blood, whole blood, processed blood, urine, saliva, stool (liquid and solid), semen or seminal fluid, cells, tissues, biopsy material, DNA, RNA, or any fluid or solid). In some embodiments, enrichment comprises washing and concentration of a biological sample, for example by allowing the biomarker-specific nanoparticles to be washed, then isolated to remove or minimize interferences prior to a biomarker characterization and measurement step.

In some embodiments, the methods described herein are used to isolate and purify a specific target (e.g., a biomarker) in a biological sample for subsequent elution and testing, or to enrich or increase the concentration of the biomarker prior to the diagnostic test.

After washing or isolating the biomarker specific particles, the particles can be dispersed, reconstituted or resuspended in a buffer such as phosphate buffered saline (i.e. PBS pH 7.2), or LC-MS/MS compatible buffer, prior to the characterization or measurement step. This means the key characterization or measurement step of the captured and enriched biomarkers by the particles occurs in a buffer system and not in the animal or human matrix which is what introduces or causes the matrix effect or bias between biomarkers measured in animal blood, plasma, serum or urine as compared to the same biomarkers measured in blood, plasma, serum or urine using the same characterization, measurement or test method or system. Wash allows sample matrix, components, proteins and cellular constituents and associated interference or matrix effect be washed away.

Enrichment is defined as complete if sufficient quantity of anayte(s) is captured for subsequent diagnostic test, e.g., quantitative, semi-quantitative, or qualitative analysis, and is defined as partial if sufficient quantity of analyte(s) or biomarker(s) is captured for subsequent semi-quantitative or qualitative analysis, or also partial if sufficient quantity of target analyte(s) or biomarker(s) and internal standard(s) is captured for quantitative, semi-quantitative or qualitative analysis by measurement methods that can use internal standards to adjust for recovery of the target analyte(s) or biomarker(s) such as LCMS and LC-MS/MS (i.e. deuterated internal standard) and HPLC (C14 or tritiated internal radioisotope internal standards).

A method is provided herein for enriching a biomarker(s) in a sample prior to a diagnostic test consisting of: a) adding a particle (e.g., nanoparticle, microparticle) to the sample; b) mixing the sample with the particle (e.g., nanoparticle, microparticle); c) incubating the particle (e.g., nanoparticle, microparticle) with the sample to bind and capture the biomarker(s) to the particle (e.g., nanoparticle, microparticle); d) separating or removing the particle (e.g., nanoparticle, microparticle) from the sample; e) saving the particle (e.g., nanoparticle, microparticle); 0 washing the particle (e.g., nanoparticle, microparticle) using an appropriate wash diluent to remove non-specific materials; g) measuring the amount, mass, molarity, concentration, or yield of biomarker(s) captured by the particle (e.g., nanoparticle, microparticle) using a qualitative, semi-quantitative or quantitative diagnostic test specific for the biomarker(s). In some embodiments, the diluent comprises water (e.g., deionized water, water for injection, saline, a buffered aqueous solution).

In some embodiments, the methods of enrichment described herein comprise a washing step. The washing step removes interferences as described herein and/or provides washed, purified, or isolated biomarker(s) of interest (e.g., a biomarker(s) as described herein). In some embodiments, the methods of enrichment described herein reduce matrix effects or species effects. In some embodiments, the methods of enrichment described herein are used prior to a diagnostic test comparing two biological samples of different origin. In some embodiments, the methods of enrichment described herein are used prior to a diagnostic test comparing an animal sample and a human sample. In some embodiments, the methods of enrichment described herein are used prior to a diagnostic test comparing a serum sample and a plasma sample. In some embodiments, the methods of enrichment described herein is used on a sample of high viscosity.

The methods of enrichment described herein can be used to mitigate, reduce or manage known pre-analytical and analytical sources of testing error due to hemolysis, lipemia, icterus, bilirubin, microfibrin clots, cellular debris, blood cells, fibrinogen, other interfering substances such as drugs, metabolites, supplements, herbal remedies, and multivitamins by allowing the biomarker(s)-specific nanoparticles to be washed or isolated to remove and minimize said interferences prior to the biomarker(s) characterization and measurement step.

In some embodiments, the methods of enrichment comprise combining of a first biological sample enriched with a biomarker(s) with a second biological sample enriched with the biomarker(s).

Provided herein is a method of measuring the amount, mass, molarity, concentration, or yield of targeted biomarker(s) captured and enriched by the particle (e.g., nanoparticle, microparticle) whereby the biomarker(s) is eluted, disassociated or freed from the particle (e.g., nanoparticle, microparticle) by the cleavage reagent described herein, e.g., by disrupting the binding interaction using elution strategies such as pH (e.g. increased pH with a base such as sodium bicarbonate, decreased pH with an acids such as acetic acid, trichloroacetic acid, sulfosalicylic acid, HCl, formic acid, and common pH elution buffers such as 100 mM glycine.HCl, pH 2.5-3.0, 100 mM citric acid, pH 3.0, 50-100 mM triethylamine or triethanolamine, pH 11.5, 150 mM ammonium hydroxide, pH 10.5), a displacer or displacing agent, competitive elution (e.g. >0.1M counter ligand or analog), ionic strength and/or chaotropic effects (e.g. NaCl, KCl, 3.5-4.0M magnesium chloride pH 7.0 in 10 mM Tris, 5M lithium chloride in 10 mM phosphate buffer pH 7.2, 2.5M sodium iodide pH 7.5, 0.2-3.0M sodium thiocyanate), surfactant, detergent, a concentrated inorganic salt, denaturing (e.g. 2-6M guanidine.HCl, 2-8M urea, 1% deoxycholate, 1% SDS), an organic solvent (e.g. alcohol, chloroform, ethanol, methanol, acetonitrile, hexane, DMSO, 10% dioxane, 50% ethylene glycol pH 8-11.5 (also chaotropic)), radiation or heat (increased temperature), conformational change, disulfide bond reducers (2-mercaptoethanol, dithiothreitol, tris(2-carboxylethyl)phosphine), enzyme inactivation, chaotropic agents (Urea, Guanidinium chloride, Lithium perchlorate), mechanical agitation, sonication, and protein digestive enzymes (pepsin, trypsin), and combinations thereof.

Unless otherwise stated, or implicit from the disclosure, any of the embodiments described in connection with any particular method or composition described herein can be used in conjunction with any of the other embodiments described herein.

The methods and compositions of the invention can be used in conjunction with any suitable assay known in the art, for example any suitable affinity assay or immunoassay known in the art including, but not limited to, protein-protein affinity assays, protein-ligand affinity assays, nucleic acid affinity assays, indirect fluorescent antibody assays (IFAS), enzyme-linked immunosorbant assays (ELISAs), radioimmunoassays (RIAs), and enzyme immunoassays (EIAs), direct or indirect assays, competitive assays, sandwich assays, CLIA or CLIA waved tests, LC-MS/MS, analytical assays, etc.

A method of both depleting sample interferences and enriching biomarkers from the same sample prior to the diagnostic test consisting of: a) add a chemical and/or biological reagent, additive, or composition (e.g., nanoparticle, microparticle) to the sample to block or deplete sample-specific interferences prior to the addition of a biomarker specific particle (e.g., nanoparticle, microparticle) to the sample; b) add a biomarker specific particle (e.g., nanoparticle, microparticle) to the sample after pre-treating or incubating the sample with the chemical and/or biological reagent, additive or composition; c) incubate the biomarker specific particle (e.g., nanoparticle, microparticle) with the sample to bind and capture the targeted biomarker(s) to the particle (e.g., nanoparticle, microparticle); d) wash the particle (e.g., nanoparticle, microparticle) or isolate it from the sample and chemical and/or biological reagent, additive or composition e) characterize the biomarker(s) captured and enriched by the particle (e.g., nanoparticle, microparticle) using a diagnostic test.

For example, in an embodiment, a particle bound to CaptAvidin would bind to biotin in a sample at neutral pH. The biotin bound to the CaptAvidin particle would release biotin when the pH is raised to 10.

Biomarkers

Described herein are methods to isolate or for isolating or enriching one or more biomarkers present in a biological sample. A “biomarker” or “biomarkers,” as referred to herein, is defined as a distinctive biological or biologically derived indicator (e.g., a metabolite) of a process, event, or condition such as aging or disease. Biomarkers may be an endogenous and/or exogenous analyte, antigen, small molecule, large molecule, drug, therapeutic agent, metabolite, xenobiotic, chemical, peptide, protein, protein digest, viral antigen, bacteria, cell, cell lysate, cell surface marker, epitope, antibody, a fragment of an antibody, IgG, IgM, IgA, IgE, IgD receptor, a ligand of a receptor, hormone, a receptor of a hormone, enzyme, a substrate of an enzyme, a single stranded oligonucleotide, a single stranded polynucleotide, a double stranded oligonucleotide, a double stranded polynucleotide, polymer, molecularly imprinted polymer, and aptamer. In some embodiments, biomarkers is an interference described herein (e.g., a substance present in a patient specimen that can alter the correct value of the result of a diagnostic test, e.g., by interfering with antibody binding, or that can increase or decrease assay signal by bridging, steric hindrance, or autoantibody mechanisms. “Interferences,” as used herein, can be, but not limited to, heterophile or heterophile-like interferences such as autoantibodies, rheumatoid factor (RF), human anti-mouse antibodies (HAMA), human anti-animal antibodies (HAAA) such as goat, rabbit, sheep, bovine, mouse, horse, pig, and donkey polyclonal and/or monoclonal antibodies, and manufacture assay-specific interference used in the test design or assay formulation, such as the chemiluminescent substrate (luminol, isoluminol, isoluminol derivatives, ABEI, ABEI derivatives, ruthenium, acridinium ester), fluorescent labels such as fluorescein or other fluorophores and dyes, capture moieties (streptavidin, neutravidin, avidin, CaptAvidin, polyA, polyDT, aptamers, antibodies, Fab, F(ab′)2, antibody fragments, recombinant proteins, enzymes, proteins, biomolecules, polymers, molecularly imprinted polymers) and their binding partners (i.e. biotin, fluorescein, PolyDT, PolyA, antigen, etc.), conjugation linkers (LC, LC-LC, PEO, PEOn), bovine serum albumin, human serum albumin, ovalbumin, gelatin, purified poly- and monoclonal IgG such as mouse, goat, sheep and rabbit, polyvinyl alcohol (PAA), polyvinylpyrrolidone (PVP), Tween-20, Tween-80, Triton X-100, triblock copolymers such as Pluronic and Tetronic, and commercially available blockers, blocking proteins and polymer-based blocking reagents such those from Surmodics and Scantibodies) typically used in the design of antibody-based diagnostic tests, non-antibody based diagnostic tests, or sample pre-treatment methods and devices for subsequent analysis by mass spectrometry (i.e. HPLC, MS, LCMS, LC-MS/MS), radioimmunoassay (RIA), enzyme-linked immunoassay (ELISA), chemiluminescence immunoassay (CLIA), molecular diagnostics, lateral flow, point-of-care (PoC), CLIA and CLIA waived tests and devices). In some embodiments, biomarkers are found in biological samples described herein.

Fibrinogen. Fibrinogen is converted during tissue and vascular injury by thrombin to fibrin, which subsequently results in the formation of a fibrin-based blood clot. In some embodiments, the particles described herein (e.g., particle-derivizatized anti-fibrinogen (e.g., mouse anti-fibrinogen)) used in the methods described herein bind and allow separation (e.g., chemical separation) of fibrinogen in whole blood. Particle binding to the clot via fibrin can be isolated and removed from the serum post-centrifugation for particle-free serum testing. In some embodiments, the biomarker is fibrinogen. In some embodiments, the methods described herein use particle-derivizatized anti-fibrinogen to remove the need for centrifugation of samples (e.g., blood samples).

For example, in some embodiments, these magnetic nanoparticles do not need to also have specificity to the clot or cellular debris to be isolated via centrifugation or the combination of centrifugation and magnetic separation in the centrifuge, their surface could be co-coated or immobilized with more than one antibody and/or antigen where one antibody or antibodies would be specific for the clot and/or cellular debris, while the other antibody(s) and/or antigen would be specific to the sample interference. In this regards the nanoparticles would specifically bind to both sample interference as well as the clot and/or cellular debris for subsequent physical separation or isolation via centrifugation or the combination of centrifugation and magnetic separation.

Use of nanoparticles specific for the clot and/or cellular debris may increase clotting rate of speed by specific binding by the magnetic nanoparticles and pulling everything to a magnetic for magnetic separation and isolation. This bead-based pellet formed by the magnetic field and strength may also accelerate the clot formation based on forced proximity of the clot or specifically captured clotting factors by the nanoparticles and subsequently the magnet.

Traumatic Brain Injury. In one embodiment, the biomarker(s) is for traumatic brain injury. There are nine (9) biomarkers associated with the severity and magnitude of acute brain injury and the integrity of the blood brain barrier (BBB), but they are present at very low circulating concentrations in blood and are very difficult to detect and quantitate using existing immunoassay technologies and test platforms. While the Banyan BTI test (FDA cleared Feb. 14, 2018) measures only 2 of these biomarkers, the methods and devices (e.g., methods of enrichment; devices for enrichment) described herein enable simultaneous measurement of all 9 biomarkers in a patient to aid in the near patient diagnosis and prognosis. Particles derivatized with capture moieties for each of the 9 biomarkers may be added to a biological sample from a patient suspected to have TBI. In some embodiments, the traumatic brain injury biomarker is selected from the group consisting of: S100B, GFAP, NLF, NFH, γ-enolase (NSE), α-II spectrin, UCH-L1, total tau, and phosphorylated tau. In some embodiments, the traumatic brain injury biomarker is selected from GFAP and UCH-L1.

In some embodiments, the methods described herein (e.g., methods of enrichment) are used to isolate or enrich the presence of one, two, three, four, five, six, seven, eight, or nine of the traumatic brain injury biomarkers selected from the group consisting of: S100B, GFAP, NLF, NFH, γ-enolase (NSE), α-II spectrin, UCH-L1, total tau, and phosphorylated tau.

Alzheimer's Disease. In one embodiment, the biomarker is for Alzheimer's Disease. There are two (2) biomarkers associated with the severity and magnitude of Alzheimer's Disease. In some embodiments, the Alzheimer's Disease biomarker is selected from the group consisting of: amyloid beta, BACE1, and soluble AP precursor protein (sAPP). In some embodiments, the Alzheimer's Disease biomarker is selected from the group consisting of: β-amyloid (1-42), phospho-tau (181p), and total-tau. In some embodiments, the methods described herein (e.g., methods of enrichment) are used to isolate or enrich the presence of one, two or three of the Alzheimer's Disease biomarkers selected from the group consisting of: amyloid beta, BACE1, and soluble AP precursor protein (sAPP). In some embodiments, the biomarker is amyloid beta, BACE1, or soluble AP precursor protein (sAPP). In some embodiments, the biomarker for Alzheimer's Disease is found in a biological sample (e.g., CSF).

Sexually Transmitted Diseases. In one embodiment, the biomarker is for a sexually transmitted disease (STD). There are at least ten (10) biomarkers characteristic of transmission of a STD. In some embodiments, the STD biomarker is a biomarker for Chlamydia, Gonorrhea, Syphilis, Trichomonas, HPV, Herpes 1 and 2, HSV, Hepatitis A, Hepatitis B, Hepatitis C, HIV 1 and 2, and HIV antibodies. In some embodiments, the methods described herein (e.g., methods of enrichment) are used to isolate or enrich the presence of one, two, three, four, five, six, seven, eight, nine, ten, eleven, twelve or thirteen STD biomarkers: Chlamydia, Gonorrhea, Syphilis, Trichomonas, HPV, Herpes 1 and 2, HSV, Hepatitis A, Hepatitis B, Hepatitis C, HIV 1 and 2, and HIV antibodies. In some embodiments, the biomarker is in urine (e.g., Chlamydia, Gonorrhea, Trichomonas). In some embodiments, the biomarker is in blood, serum, or plasma (e.g., Syphilis, HPV, Herpes 1 and 2, HSV, Hepatitis A, Hepatitis B, Hepatitis C, HIV 1 and 2, and HIV antibodies).

Bacterial Infection. In one embodiment, the biomarker is for a bacterial infection, e.g., sepsis. The current gold standard test for bacterial infection is blood culture which can take 24-48 hours before a positive result can be reflexed to a confirmatory test such as molecular diagnostics. Described herein are methods to rule-in/rule-out bacterial infection in as little as 30 minutes or less where time is critical to successfully treat patients to prevent or manage sepsis, for example in 60 minutes or less (e.g., 50 minutes, 40 minutes, 30 minutes, 20 minutes or less). There are at least thirty (30) biomarkers characteristic of bacterial infection. In some embodiments, the bacterial biomarker is selected from the group consisting of a biomarker for sepsis-causing species of bacteria (e.g., Enterococcus faecium, Escherichia coli, Klebsiella pneumoniae, Pseudomonas aeruginosa, and Staphylococcus aureus). In some embodiments, the biomarker is a biomarker for Enterococcus faecium, Escherichia coli, Klebsiella pneumoniae, Pseudomonas aeruginosa, and Staphylococcus aureus. In some embodiments, the biomarker is a biomarker for a gram positive or gram negative bacteria. In some embodiments, the biomarker is a biomarker for a yeast pathogen (e.g., a yeast pathogen associated with bloodstream pathogens).

In some embodiments, the gram positive bacteria is: Enterococcus, Listeria monocytogenes, Staphylococcus, Staphylococcus aureus, Streptococcus, Streptococcus agalactiae, Streptococcus pneumoniae, or Streptococcus pyogenes.

In some embodiments, the gram negative bacteria is: Acinetobacter baumannii, Haemophilus influenza, Neisseria meningitides, Pseudomonas aeruginosa, Enterobacteriaceae, Enterobacter cloacae complex, Escherichia coli, Klebsiella oxytoca, Klebsiella pneumoniae, Proteus, or Serratia marcescens.

In some embodiments, the yeast pathogen is: Candida albicans, Candida glabrata, Candida krusei, Candida parapsilosis, Candida tropicalis.

In some embodiments, followed by mass spectrometry method. Method is envisioned where a cleavage agent (e.g., reducing agent (e.g., DTT or TCEP)) is added to a bacteria-particle bound complex to cleave the linker (i.e., linker conjugating particle to surface capture moiety). The resultant bacterial is grown in culture or analyzed by MALDI-TOF mass spectrometry or a molecular diagnostic method.

Method is envisioned where a cleavage agent (e.g., reducing agent (e.g., DTT or TCEP)) is added to a bacteria-particle bound complex to cleave the linker (i.e., linker conjugating particle to surface capture moiety). The resultant bacterial is grown in culture or analyzed by MALDI-TOF mass spectrometry or a molecular diagnostic method.

Thyroid Function. TSH concentrations are measured as part of a thyroid function test in patients suspected of having an excess (hyperthyroidism) or deficiency (hypothyroidism) of thyroid hormones. The methods described herein in some embodiments are used to evaluate thyroid function. In some embodiments, the biomarker is an antigen (e.g., TSH). In some embodiments, the capture moiety is an autoantibody (e.g., free autoantibody, complexed autoantibody) with specificity to the antigen (e.g., TSH).

Cardiac Function. The methods described herein in some embodiments are used to evaluate cardiac function. An increased level of troponin circulating in blood is a biomarker for heart disorders, e.g., myocardial infarction. Cardiac I and T are specific indicators of heart muscle damage. Subunits of troponin are also markers for cardiac health. Specifically cTnI and cTnT are biomarkers for acute myocardial infarction (AMI) for example type 1 and 2 myocardial infarction, unstable angina, post-surgery myocardium trauma and related diseases. In some embodiments, the biomarker is free cTnI, free cTnT, binary cTnI-TnC, or ternary cTnI-TnC-TnT. In some embodiments, the biomarker is an indicator for heart failure. In some embodiments, the biomarker is an indicator for stroke (e.g., as described in https://www.ahaj ournals. org/doi/10.1161/STROKEAHA.117.017076 and https://www.360dx.com/business-news/roche-test-helps-differentiate-bleeding-risk-stroke-risk-patients-considering#.W1jz0thKhcA, which are incorporated by reference in their entirety). In some embodiments, the biomarker is an indicator for fibrosis (e.g., as described in http://www.onlinejacc.org/content/65/22/2449, which is incorporated by reference in its entirety). In some embodiments, the biomarker is for diagnosis of acute coronary syndrome (ACS). In some embodiments, the biomarker is for Cardiac Troponin (I, I-C, I-C-T, T) and other cardiac troponin fragments, Natriuretic Peptides (BNP, ANP, CNP), N-terminal fragments (i.e. NT-proBNP, NT-proCNP), glycosylated, non-glycosylated, CRP, Myoglobin, Creatinine kinase (CK), CK-MB, sST2, GDF-15, Galectin-3.

Obstructive Sleep Apnea (OSA). Urocortin III peptide, Uromodulin peptide, Orosomucoid 1 peptide, and Kallikrein 1 peptide may be used to diagnose OSA. Methods and biomarkers for diagnosing OSA have been described in US 2006/0029980, US 2016/0161489, U.S. Pat. Nos. 8,999,658 and 9,435,814, the contents of which are each incorporated by reference in their entireties. Biomarkers for OSA are also described in De Luca Canto et al., Sleep Med. Rev. 2015 Oct.; 23: 28-45, the contents of which are incorporated by reference in its entirety. The methods described herein in some embodiments are directed to improved methods for diagnosing OSA. In some embodiments, the biomarker for OSA is Urocortin III peptide, Uromodulin peptide, Orosomucoid 1 peptide, Kallikrein 1 peptide, IL-6, IL-10, high sensitivity C-reactive protein, or a combination thereof. In some embodiments, the biomarker (e.g., Uromodulin peptide) is in low abundance in the sample. In some embodiments, the capture moiety is an antibody with specificity to Urocortin III peptide, Uromodulin peptide, Orosomucoid 1 peptide, Kallikrein 1 peptide, or a combination thereof.

In an aspect, described herein is a method of detecting Urocortin III peptide, Uromodulin peptide, Orosomucoid 1 peptide, Kallikrein 1 peptide, IL-6, IL-10, high sensitivity C-reactive protein, or a combination thereof, in a patient, said method comprising: a. obtaining a sample from a human patient; and b. detecting whether Urocortin III peptide, Uromodulin peptide, Orosomucoid 1 peptide, Kallikrein 1 peptide, IL-6, IL-10, high sensitivity C-reactive protein, or a combination thereof is present in the sample with a plurality of particles and detecting binding between Urocortin III peptide, Uromodulin peptide, Orosomucoid 1 peptide, Kallikrein 1 peptide, IL-6, IL-10, high sensitivity C-reactive protein, or a combination thereof, and the plurality of particles. In an aspect, described herein is a method of diagnosing obstructive sleep apnea in a patient, said method comprising: a. obtaining a sample from a human patient; and b. detecting whether Urocortin III peptide, Uromodulin peptide, Orosomucoid 1 peptide, Kallikrein 1 peptide, IL-6, IL-10, high sensitivity C-reactive protein, or a combination thereof is present in the sample by contacting the sample with a plurality of particles and detecting binding between Urocortin III peptide, Uromodulin peptide, Orosomucoid 1 peptide, Kallikrein 1 peptide, IL-6, IL-10, high sensitivity C-reactive protein, or a combination thereof, and the plurality of particles; and c. diagnosing the patient with obstructive sleep apnea when the presence of Urocortin III peptide, Uromodulin peptide, Orosomucoid 1 peptide, Kallikrein 1 peptide, IL-6, IL-10, high sensitivity C-reactive protein in the sample is detected. In some embodiments, the patient is a human. In some embodiments, the human is under the age of about 9 to about 2 years of age. In some embodiments, the method comprises detecting the presence or absence of two or more of the group consisting of Urocortin III peptide, Uromodulin peptide, Orosomucoid 1 peptide, Kallikrein 1 peptide, IL-6, IL-10, and high sensitivity C-reactive protein, concurrently.

In some embodiments, the sample is a urine sample. In some embodiments, the sample is a blood sample. In some embodiments, a multi-marker approach (uromodulin, urocortin-3, oro-somucoid-1, and kallikrein) in urine samples in pediatric patients is used. In some embodiments, the measurements of urinary Lipocalin-type prostaglandin D synthase (L-PGDS) concentrations in an adult population is used as a marker to identify patients with severe OSA. In some embodiments, an oxidative stress multi-biomarker approach (L-PGDS, F2-isoprostane, and others) in adult populations may be used to predict OSA. In some embodiments, biomarkers for OSA are evaluated using chromatography and/or MS methods. In some embodiments, proteins and metabolites, including lipid profiles, adrenergic/dopaminergic bio-markers and derivatives, amino acids, oxidative stress biomarkers, and other micro-molecules are used in the diagnosis and/or effective treatment OSA. In some embodiments, IL-6 and/or hsCRP are used as biomarkers to discriminate OSA patients with and without morbidity in adults. In some embodiments, Myeloid-related protein (MRP) 8/14 is used to discriminate OSA patients with and without morbidity in children. In some embodiments, serum sLOX-1 levels are used as an independent predictor for the presence of OSA. In some embodiments, increased serum YKL-40 concentration is used as an independent risk factor for the presence of OSA. In some embodiments, elevated serum chemerin levels are used as an independent predicting marker of the presence and severity of OSA.

In some embodiments, the sample is urine and the OSA biomarker is uromodulin, urocortin-3, oro-somucoid-1, kallikrein, lipocalin-type prostaglandin D synthase (L-PGDS), F2-isoprostane, or a combination thereof. In some embodiments, the sample is serum or blood based and the OSA biomarker is IL-6, hsCRP, sLOX-1, YKL-40, myeloid-related protein (MRP) 8/14, chemerin, or a combination thereof.

In some embodiments, the accuracy and precision by being able to test large sample volumes (i.e. 1 mL, 10 mL, 100 mL, 1000 mL, etc.) to improve likelihood of detection of very dilute or low concentration biomarker(s), as well as very small sample volumes (i.e. neonates, pediatrics, elderly) which typically are untestable today or require sample dilution before testing which compromises test sensitivity, accuracy and precision. In some embodiments, the biological sample is in a 1 mL, 10 mL, 100 mL, 1000 mL or greater volume. In some embodiments, the biological sample is in a 0.5 mL, 0.25 mL, 0.1 mL, 0.05 mL or lesser volume.

Also provided herein is a method for using particle sample pre-treatment to aid in enrichment of biomarkers prior to a diagnostic test by allowing a wash step or particle isolation followed by selective release or elution of the captured biomarker(s), or selective release or elution of the capture moiety-biomarker complex, from the particles prior to the biomarker characterization step or test method.

The use of a “cleavage reagent or “releasing agent” that will disrupt the bond between the capture moiety on the particle surface and the biomarker, e.g., acidic or basic pH, high molarity salt, sugar, chemical displacer, detergent, surfactant, and/or chelating agent, or combination thereof, without displacing or eluting the capture moiety but only the biomarker. After washing or isolating the particles from the sample matrix with magnet(s), the particles can subsequently be treated with an elution solution containing a releasing agent(s) to selectively release the biomarker into solution. The particles can be quickly (less than 2 minutes; ideally less than 30 seconds) isolated to the side(s) and/or bottom of the sample device (vial, test tube, other) to form an essentially particle-free sample supernatant. The particle-free supernatant can be subsequently aspirated without disrupting the pellet comprising particles and dispensed into a separate transfer tube or injected directly onto the analytical system (i.e. LC-MS/MS or MALDI-TOF) for testing of the biomarker(s).

For example, the cleavage reagent or releasing agent described herein disrupt the binding interaction or cleavable bond as described herein between the particles described herein and a capture moiety described herein, e.g., using elution strategies such as pH (e.g. increased pH with a base such as sodium bicarbonate, decreased pH with an acids such as acetic acid, trichloroacetic acid, sulfosalicylic acid, HCl, formic acid, and common pH elution buffers such as 100 mM glycine.HCl, pH 2.5-3.0, 100 mM citric acid, pH 3.0, 50-100 mM triethylamine or triethanolamine, pH 11.5, 150 mM ammonium hydroxide, pH 10.5), a displacer or displacing agent, competitive elution (e.g. >0.1M counter ligand or analog), ionic strength and/or chaotropic effects (e.g. NaCl, KCl, 3.5-4.0M magnesium chloride pH 7.0 in 10 mM Tris, 5M lithium chloride in 10 mM phosphate buffer pH 7.2, 2.5M sodium iodide pH 7.5, 0.2-3.0M sodium thiocyanate), surfactant, detergent, a concentrated inorganic salt, denaturing (e.g. 2-6M guanidine.HCl, 2-8M urea, 1% deoxycholate, 1% SDS), an organic solvent (e.g. alcohol, chloroform, ethanol, methanol, acetonitrile, hexane, DMSO, 10% dioxane, 50% ethylene glycol pH 8-11.5 (also chaotropic)), radiation or heat (increased temperature), conformational change, disulfide bond reducers (2-mercaptoethanol, dithiothreitol, tris(2-carboxylethyl)phosphine), enzyme inactivation, chaotropic agents (Urea, Guanidinium chloride, Lithium perchlorate), mechanical agitation, sonication, and protein digestive enzymes (pepsin, trypsin), and combinations thereof.

### Method of Characterization

Described herein are methods for depleting and/or enriching biomarkers for subsequent characterization or diagnostic testing. Characterization of a biomarker described herein (e.g., interference) includes identification and/or quantification of a biomarker described herein (e.g., interference described herein).

Characterization of a biomarker described herein can include signal amplification technologies (e.g., chemiluminescence, fluorescence, metal-enhanced fluorescence) to improve sensitivity of detection.

For example, biomarker characterization can use singleplex or multiplex characterization approaches where detection can be visual (colored beads) or measure a specific signal (UV/vis absorbance, fluorescence, chemiluminescence, electrochemiluminescence, turbidimetric, etc.) using labeled particles.

In some embodiments, the presence of biomarker(s) is determined by MALDI-MS. In some embodiments, the presence of biomarker(s) is determined by a molecular diagnostic method. In some embodiments, the presence of biomarker(s) is determined by an immunoassay.

### Particles of the Invention

Described herein are particles for the isolation, depletion and/or enrichment of biological samples. In some embodiments, the particles comprise a non-cleavable bond and a capture moiety (e.g., a particle surface functionalized to present one or more capture moieties. In some embodiments, the particles described herein comprise a capture moiety (e.g., a capture moiety with high specificity to a biomarker(s) described herein). In some embodiments, the particles described herein (e.g., the surface of the particles described herein, the particle surface not bound to a capture moiety described herein) are inert (e.g. do not exhibit significant binding to a biomarker(s) described herein). In some embodiments, the particles described herein can be used in the diagnostic tests described herein without further modification to the particle or the diagnostic test. In some embodiments, the particles described herein can be added to and removed from a sample without altering the sample (e.g., without adding or removing an additional biomarker(s) (e.g., an interference). Particles are also referred to herein as beads.

The particles described herein are sufficiently small with a mean diameter from 0.050 micrometers up to 3.00 micrometers, or preferably from 0.100 micrometers to 1.1 micrometers in diameter, or still more preferably 0.200 micrometers to 0.600 micrometers, or even more preferably from 0.100 micrometers to 0.500 micrometers in diameter. In some embodiments, the particle is 5 nm to 100 nm in diameter. In some embodiments, the particle is 50 nm to 100 nm in diameter. In some embodiments, the particle is 100 nm to 500 nm in diameter.

In some embodiments, the particles described herein (e.g., microparticle, nanoparticle) comprise a core or support, wherein the core or support is a paramagnetic or superparamagnetic material selected from the group consisting of iron oxide, ferromagnetic iron oxide, Fe₂O₃, and Fe₃O₄, maghemite, or combinations thereof.

In some embodiments, the particle surface comprises an organic polymer or copolymer, wherein the organic polymer or copolymer is hydrophobic. In some embodiments, the particle (e.g., nanoparticle, microparticle) surface comprises an organic polymer or copolymer such as a material selected from the group consisting of, but not limited to, ceramic, glass, a polymer, a copolymer, a metal, latex, silica, a colloidal metal such as gold, silver, or alloy, polystyrene, derivatized polystyrene, poly(divinylbenzene), styrene-acylate copolymer, styrene-butadiene copolymer, styrene-divinylbenzene copolymer, poly(styrene-oxyethylene), polymethyl methacrylate, polymethacrylate, polyurethane, polyglutaraldehyde, polyethylene imine, polyvinylpyrrolidone, polyvinyl alcohol, polyacrylic acid, N,N′-methylene bis-acrylamide, polyolefeins, polyethylene, polypropylene, polyvinylchloride, polyacrylonitrile, polysulfone, poly(ether sulfone), pyrolized materials, block copolymers, and copolymers of the foregoing, silicones, or silica, methylol melamine, a biodegradable polymer such as dextran or poly(ethylene glycol)-dextran (PEG-DEX), or combinations thereof.

In some embodiments, the particle surface comprises functional groups or a plurality of functional groups for covalent attachment (coupling, conjugation or binding) of capture moiety thereof such as carboxyl, tosyl, epoxy, amine, sulfhydryl, hydroxyl, ester, methyl chloride, and maleimide, click chemistry functionality [Copper(I)-Catalyzed Azide-Alkyne Cycloaddition (CuAAC), Strain-promoted Azide-Alkyne Cycloaddition (SPAAC), Strain-promoted Alkyne-Nitrone Cycloaddition (SPANC), and Reactions of Strained Alkenes such as Alkene and Azide [3+2] cycloaddition, Alkene and Tetrazine inverse-demand Diels-Alder, and Alkene and Tetrazole photoclick reaction], hydrazone-based coupling functionality such as S-HyNic (succinimidyl-6-hydrazino-nicotinamide) and S-4FB (N-succinimidyl-4-formylbenzamide) heterobifunctional crosslinkers, and photoreactive chemistries.

As used herein, “blocker” refers to a protein, polymer, surfactant, detergent, or combinations thereof. In some embodiments, the binding of a capture moiety on a particle described herein (e.g., nanoparticle, microparticle) is blocked with a blocker such as a protein, polymer, surfactant, detergent, or combinations thereof. The blocker is selected from the group consisting of a protein such as albumin, bovine serum albumin, human serum albumin, ovalbumin, gelatin, casein, acid hydrolyzed casein, gama globulin, purified IgG, animal serum, polyclonal antibody, and monoclonal antibody, a polymer such as polyvinyl alcohol (PVA) and polyvinylpyrrolidone (PVP), a combination of a protein and polymer, a peptide, a PEGylation reagent such as (PEO)n-NHS or (PEO)n-maleimide, a triblock copolymer such as Pluronic F108, F127, and F68, a non-ionic detergent such as Triton X-100, polysorbate 20 (Tween-20), and Tween 80 (non-ionic), a zwitterionic detergent such CHAPS, a ionic detergent such as sodium dodecyl sulfate (SDS), deoxycholate, cholate, and sarkosyl, a surfactant, a sugar such as sucrose, and a commercial blocker such as Heterophilic Blocking Reagent (Scantibodies), MAK33 (Roche Diagnostics), Immunoglobulin Inhibiting Reagent (IIR) (Bioreclamation), Heteroblock (Omega Biologicals), Blockmaster (JSR), TRU Block (Meridian Life Sciences), and StabilCoat® & StabilGuard® (Surmodics). In some embodiments, the blocker is bound to a particle described herein (e.g., covalently bound, non-covalently bound). In some embodiments, the blocker is not bound (e.g., covalently bound, non-covalently bound) to a particle described herein.

Capture Moieties. Provided herein are particles comprising one or more capture moieties that bind an interference as described herein, or a biomarker(s) as described here. As referred to herein, “capture moiety” is selected from the group consisting of an antibody, a binding fragment of an antibody, a IgG, a IgM, a IgA, IgE, IgD a receptor, a ligand of a receptor, a hormone, a receptor of a hormone, an enzyme, a substrate of an enzyme, a single stranded oligonucleotide, a single stranded polynucleotide, a double stranded oligonucleotide, a double stranded polynucleotide, an antigen, a peptide, a polymer, a molecularly imprinted polymer, an aptamer, and a protein.

In some embodiments, the capture moiety is a protein. A protein can be, for example, a monomer, a dimer, a multimer, or a fusion protein. In specific embodiments, the protein comprises at least one of an albumin such as, for example, antibody, a fragment of an antibody, BSA, ovalbumin, a fragment of BSA, a fragment of ovalbumin, mouse IgG, polymerized mouse IgG, antibody fragments (Fc, Fab, F(ab′)2) and different subclasses (IgG1, IgG2a, IgG2b, IgG3, IgE, IgD) of mouse IgG to target HAMA and RF interference mechanisms, purified animal polyclonal antibodies (i.e. bovine, goat, mouse, rabbit, sheep) to target HAAA interference, streptavidin, ALP, HRP, BSA (conjugated to isoluminol, ruthenium, acridinium) to target MASI interference or mixtures thereof.

In at least one embodiment, the present invention provides a binding surface with two or more different capture moieties.

Generation of Capture Moieties. In an aspect, provided is a method for making a capture moiety, the method comprising the production or generation of complex-specific or conformation-specific antibodies to free autoantibodies or autoantibody complexes. Free autoantibodies are autoantibodies that are not already complexed to their antigen target. Complexed autoantibodies are autoantibodies that have formed a complex with their antigen target.

In an aspect, provided is a method for making a capture moiety, the method comprising the production or generation of complex-specific or conformation-specific antibodies to autoantibody complexes like MTSH. In some embodiments, the autoantibody is triiodothyronine (T3) or thyroxine (T4). In some embodiments, the autoantibody complex is MTSH. For example, complex-specific or conformation-specific antibodies can be raised to autoantibody complexes like MTSH, which can be purified from human serum and used as the capture moiety. In this way the antibodies generated would only have specificity to hIgG or hIgM complexes with TSH. MTSH can be purified based on techniques and published methods or by someone skilled in the art of protein biochemistry and purification. In some embodiments, patients with autoimmune disease who have the greatest likelihood of autoantibody assay interference are used to produce or generate autoantibodies. For example, see the HyTest SES assay for BNP, WO2014114780, WO2016113719 and WO2016113720, the references of which are cited in their entirety.

Thyroid-specific Autoantibodies. For example, in an embodiment, the autoantibody is an anti-thyroid autoantibody (e.g., anti-thyroid peroxidase antibody, thyrotropin receptor antibodies, thyroglobulin antibodies). Anti-thyroid autoantibodies are autoantibodies targeted against one or more components on the thyroid.

In some embodiments, the autoantibody is a free autoantibody (e.g., thyrotropin (TSH).

In some embodiments, the autoantibody is a complexed autoantibody (e.g., MTSH). In some embodiments, the capture moieties described herein are antibodies generated with specificity to complexed autoantibodies or with confirmation specificity to the hIgG and/or hIgM already bound to its antigen target such as MTSH. In some embodiments, the autoantibody is to T3 amd T4.

Itemized below is a nonlimiting list of substances that may function as one, or alternatively as the other, member of a binding pair consisting of analyte binder (capture moiety) and analyte, depending on the application for which an affinity assay is to be designed. Such substances can be used, for example, as capture moieties (analyte binders) or can be used to generate capture moieties (e.g., by employing them as haptens/antigens to generate specific antibodies) that can be used with the invention. Affinity assays, including immunoassays, can be designed in accordance with the invention to detect the presence and/or level of such substances where they are analytes in a sample. In a specific embodiment, the analyte-binding capture moieties of the invention can be used to detect these substances as analytes in a sample. Alternatively, the substances listed below can be associated with the solid phase support surface in accordance with the invention, and used to capture molecules that interact with them (such as, for example, antibodies or fragments thereof specific for the listed substances, binding proteins, or enzymes).

A nonlimiting list of substances that may function as one, or alternatively as the other, member of a binding pair consisting of analyte binder (capture moiety) and analyte includes: inducible nitric oxide synthase (iNOS), CA19-9, IL-1a, IL-1β, IL-2, IL-3, IL-4, IL-t, IL-5, IL-7, IL-10, IL-12, IL-13, sIL-2R, sIL-4R, sIL-6R, SIV core antigen, IL-1RA, TNF-α, IFN-gamma, GM-CSF; isoforms of PSA (prostate-specific antigen) such as PSA, pPSA, BPSA, in PSA, non ai-antichymotrypsin-complexed PSA. α₁-antichymotrypsin-complexed PSA, prostate kallikreins such as hK2, hK4, and hK15, ek-rhK2, Ala-rhK2, TWT-rhK2, Xa-rhK2, HWT-rhK2, and other kallikreins; HIV-1 p24; ferritin, L ferritin, troponin I, BNP, leptin, digoxin, myoglobin, B-type natriuretic peptide or brain natriuretic peptide (BNP), NT-proBNP, CNP, NT-proCNP(1-50), NT-CNP-53(51-81), CNP-22(82-103), CNP-53(51-103), atrial natriuretic peptide (ANP); human growth hormone, bone alkaline phosphatase, human follicle stimulating hormone, human leutinizing hormone, prolactin; human chorionic gonadotrophin (e.g., CGa, CGβ; soluble ST2, thyroglobulin; anti-thyroglobulin; IgE, IgG, IgG1, IgG2, IgG3, IgG4, B. anthracis protective antigen, B. anthracis lethal factor, B. anthracis spore antigen, F. tularensis LPS, S. aureas enterotoxin B, Y. pestis capsular F1 antigen, insulin, alpha fetoprotein (e.g., AFP 300), carcinoembryonic antigen (CEA), CA 15.3 antigen, CA 19.9 antigen, CA 125 antigen, HAV Ab, HAV Igm, HBc Ab, HBc Igm, HIV1/2, HBsAg, HBsAb, HCV Ab, anti-p53, histamine; neopterin; s-VCAM-1, serotonin, sFas, sFas ligand, sGM-CSFR, sICAM-1, thymidine kinase, IgE, EPO, intrinsic factor Ab, haptoglobulin, anti-cardiolipin, anti-dsDNA, anti-Ro, Ro, anti-La, anti-SM, SM, anti-nRNP, antihistone, anti-Scl-70, Scl-70, anti-nuclear antibodies, anti-centromere antibodies, SS-A, SS-B, Sm, U1-RNP, Jo-1, CK, CK-MB, CRP, ischemia modified albumin, HDL, LDL, oxLDL, VLDL, troponin T, troponin I, troponin C, microalbumin, amylase, ALP, ALT, AST, GGT, IgA, IgG, prealbumin, anti-streptolysin, chlamydia, CMV IgG, toxo IgG, toxo IgM, apolipoprotein A, apolipoprotein B, C3, C4, properdin factor B, albumin, α₁-acid glycoprotein, α₁-antitrypsin, α₁-microglobulin, α₂-macroglobulin, anti-streptolysin O, antithrombin-III, apolipoprotein A1, apolipoprotein B, β₂-microglobulin, ceruloplasmin, complement C3, complement C4, C-reactive protein, DNase B, ferritin, free kappa light chain, free lambda light chain, haptoglobin, immunoglobulin A, immunoglobulin A (CSF), immunoglobulin E, immunoglobulin G, immunoglobulin G (CSF), immunoglobulin G (urine), immunoglobulin G subclasses, immunoglobulin M, immunoglobulin M (CSF), kappa light chain, lambda light chain, lipoprotein (a), microalbumin, prealbumin, properdin factor B, rheumatoid factor, ferritin, transferrin, transferrin (urine), rubella IgG, thyroglobulin antibody, toxoplasma IgM, toxoplasma IgG, IGF-I, IGF-binding protein (IGFBP)-3, hepsin, pim-1 kinase, E-cadherein, EZH2, and a-methylacyl-CoA racemase, TGF-beta, IL6SR, GAD, IA-2, CD-64, neutrophils CD-64, CD-20, CD-33, CD-52, isoforms of cytochrome P450, s-VCAM-1, sFas, sICAM, hepatitis B surface antigen, thromboplastin, HIV p24, HIV gp41/120, HCV C22, HCV C33, hemoglobin A1c, and GAD65, IA2, vitamin D, 25-OH vitamin D, 1,25(OH)2 vitamin D, 24,25(OH)2 vitamin D, 25,26(OH)2 vitamin D, 3-epimer of vitamin D, FGF-23, sclerostin, procalcitonin, calcitonin, c. dificille toxin A&B, h. pylori, HSV-1, HSV2.

Suitable substances that may function as one, or alternatively as the other, member of a binding pair consisting of analyte binder (capture moiety) and analyte, depending on the application for which an affinity assay is to be designed, and that can be used with the present invention also include moieties, such as for example antibodies or fragments thereof, specific for any of the WHO International Biological Reference Preparations held and, characterized, and/or distributed by the WHO International Laboratories for Biological Standards (available at http:/www.who.int/bloodproducts/re_materials, updated as of Jun. 30, 2005, which lists substances that are well known in the art; the list is herein incorporated by reference).

A partial list of such suitable international reference standards, identified by WHO code in parentheses following the substance, includes: human recombinant thromboplastin (rTF/95), rabbit thromboplastin (RBT/90), thyroid-stimulating antibody (90/672), recombinant human tissue plasminogen activator (98/714), high molecular weight urokinase (87/594), prostate specific antigen (96/668), prostate specific antigen 90:10 (96/700); human plasma protein C (86/622), human plasma protein S (93/590), rheumatoid arthritis serum (W1066), serum amyloid A protein (92/680), streptokinase (00/464), human thrombin (01/580), bovine combined thromboplastin (OBT/79), anti-D positive control intravenous immunoglobulin (02/228), islet cell antibodies (97/550), lipoprotein a (IFCC SRM 2B), human parvovirus B19 DNA (99/800), human plasmin (97/536), human plasminogen-activator inhibitor 1 (92/654), platelet factor 4 (83/505), prekallikrein activator (82/530), human brain CJD control and human brain sporadic CJD preparation 1 and human brain sporadic CJD preparation 2 and human brain variant CJD (none; each cited in WHO TRS ECBS Report No. 926, 53.sup.rd Report, brain homogenate), human serum complement components C1q, C4, C5, factor B, and whole functional complement CH50 (W1032), human serum immunoglobulin E (75/502), human serum immunoglobulins G, A, and M (67/86), human serum proteins albumin, alpha-1-antitrypsin, alpha-2-macroglobulin, ceruloplasmin, complement C3, transferrin (W1031), anti-D negative control intravenous immunoglobulin (02/226), hepatitis A RNA (00/560), hepatitis B surface antigen subtype adw2 genotype A (03/262 and 00/588), hepatitis B viral DNA (97/746), hepatitis C viral RNA (96/798), HIV-1 p24 antigen (90/636), HIV-1 RNA (97/656), HIV-1 RNA genotypes (set of 10 I01/466), human fibrinogen concentrate (98/614), human plasma fibrinogen (98/612), raised A2 hemoglobin (89/666), raised F hemoglobin (85/616), hemoglobincyanide (98/708), low molecular weight heparin (85/600 and 90/686), unfractionated heparin (97/578), blood coagulation factor VIII and von Willebrand factor (02/150), human blood coagulation factor VIII concentrate (99/678), human blood coagulation factor XIII plasma (02/206), human blood coagulation factors II, VII, IX, X (99/826), human blood coagulation factors II and X concentrate (98/590), human carcinoembryonic antigen (73/601), human C-reactive protein (85/506), recombinant human ferritin (94/572), apolipoprotein B (SP3-07), beta-2-microglobulin (B2M), human beta-thromboglobulin (83/501), human blood coagulation factor IX concentrate (96/854), human blood coagulation factor IXa concentrate (97/562), human blood coagulation factor V Leiden, human gDNA samples FV wild type, FVL homozygote, FVL heterozygote (03/254, 03/260, 03/248), human blood coagulation factor VII concentrate (97/592), human blood coagulation factor VIIa concentrate (89/688), human anti-syphilitic serum (HS), human anti-tetanus immunoglobulin (TE-3), human antithrombin concentrate (96/520), human plasma antithrombin (93/768), human anti-thyroglobulin serum (65/93), anti-toxoplasma serum (TOXM), human anti-toxoplasma serum (IgG) (01/600), human anti-varicella zoster immunoglobulin (W1044), apolipoprotein A-1 (SP1-01), human anti-interferon beta serum (G038-501-572), human anti-measles serum (66/202), anti-nuclear ribonucleoprotein serum (W1063), anti-nuclear-factor (homogeneous) serum (66/233), anti-parvovirus B19 (IgG) serum (91/602), anti-poliovirus serum Types 1, 2, 3 (66/202), human anti-rabies immunoglobulin (RAI), human anti-rubella immunoglobulin (RUBI-1-94), anti-smooth muscle serum (W1062), human anti-double-stranded DNA serum (Wo/80), human anti-E complete blood-typing serum (W1005), human anti-echinococcus serum (ECHS), human anti-hepatitis A immunoglobulin (97/646), human anti-hepatitis B immunoglobulin (W1042), human anti-hepatitis E serum (95/584), anti-human platelet antigen-1a (93/710), anti-human platelet antigen-5b (99/666), human anti-interferon alpha serum (B037-501-572), human alphafetoprotein (AFP), ancrod (74/581), human anti-A blood typing serum (W1001), human anti-B blood typing serum (W1002), human anti-C complete blood typing serum (W1004), anti-D (anti-RhO) complete blood-typing reagent (99/836), human anti-D (anti-RhO) incomplete blood-typing serum (W1006), and human anti-D immunoglobulin (01/572).

Other examples of suitable substances that may function as one, or alternatively as the other, member of a binding pair consisting of analyte binder (capture moiety) and analyte, depending on the application for which an affinity assay is to be designed include compounds that can be used as haptens to generate antibodies capable of recognizing the compounds, and include but are not limited to, any salts, esters, or ethers, of the following: hormones, including but not limited to progesterone, estrogen, and testosterone, progestins, corticosteroids, and dehydroepiandrosterone, and any non-protein/non-polypeptide antigens that are listed as international reference standards by the WHO. A partial list of such suitable international reference standards, identified by WHO code in parentheses following the substance, includes vitamin B12 (WHO 81.563), folate (WHO 95/528), homocystein, transcobalamins, T4/T3, and other substances disclosed in the WHO catalog of International Biological Reference Preparations (available at the WHO website, for example at page http://www.who.int/bloodproducts/ref materials/, updated Jun. 30, 2005), which is incorporated herein by reference. The methods and compositions described herein can comprise one or more of the aforementioned WHO reference standards or a mixture containing a reference standard.

Other examples of substances that may function as one, or alternatively as the other, member of a binding pair consisting of analyte binder (capture moiety) and analyte, depending on the application for which an affinity assay is to be designed include drugs of abuse. Drugs of abuse include, for example, the following list of drugs and their metabolites (e.g., metabolites present in blood, in urine, and other biological materials), as well any salts, esters, or ethers, thereof: heroin, morphine, hydromorphone, codeine, oxycodone, hydrocodone, fentanyl, demerol, methadone, darvon, stadol, talwin, paregoric, buprenex; stimulants such as, for example, amphetamines, methamphetamine; methylamphetamine, ethylamphetamine, methylphenidate, ephedrine, pseudoephedrine, ephedra, ma huang, methylenedioxyamphetamine (MDS), phentermine, phenylpropanolamine; amiphenazole, bemigride, benzphetamine, bromatan, chlorphentermine, cropropamide, crothetamide, diethylpropion, dimethylamphetamine, doxapram, ethamivan, fencamfamine, meclofenoxate, methylphenidate, nikethamide, pemoline, pentetrazol, phendimetrazine, phenmetrazine, phentermine, phenylpropanolamine, picrotoxine, pipradol, prolintane, strychnine, synephrine, phencyclidine and analogs such as angel dust, PCP, ketamine; depressants such as, for example, barbiturates, gluthethimide, methaqualone, and meprobamate, methohexital, thiamyl, thiopental, amobarbital, pentobarbital, secobarbital, butalbital, butabarbital, talbutal, and aprobarbital, phenobarbital, mephobarbital; benzodiazapenes such as, for example, estazolam, flurazepam, temazepam, triazolam, midazolam, alprazolam, chlordiazepoxide, clorazepate, diazepam, halazepam, lorazepam, oxazepam, prazepam, quazepam, clonazepam, flunitrazepam; GBH drugs such as gamma hydroxyl butyric acid and gamma butyrolactone; glutethimide, methaqualone, meprobamate, carisoprodol, zolpidem, zaleplon; cannabinoid drugs such as tetrahydracannabinol and analogs; cocaine, 3-4 methylenedioxymethamphetamine (MDMA); hallucinogens such as, for example, mescaline and LSD.

### EXAMPLES

### Example 1: Low Abundance Biomarker Enrichment

Very low levels of a biomarker in 40 mL PBS, either 0.0195 μIU TSH/mL or 0.497 pg PTH/mL) were enriched using 550 nm superparamagnetic nanoparticles coated with Streptavidin and subsequently coated with biotinylated anti-TSH antibody (VERAPREP Concentrate TSH reagent) or biotinylated anti-PTH monoclonal antibody (VERAPREP Concentrate PTH reagent).

In the first study, VERAPREP Concentrate TSH reagent was prepared by coating 550 nm VERAPREP Biotin with biotinylated anti-TSH capture antibody. 0.08 mL TSH antigen (10 μIU/mL ELISA calibrator) was diluted to 0.0195 μIU/mL in 41 mL PBS buffer below the Functional Sensitivity (<0.054 μIU/mL) of the DRG TSH Ultrasensitive ELISA (Part No. EIA-1790, Lot No. RN58849), and 1 mL was saved as the Baseline Sample (prior to enrichment). The 40 mL sample was processed using a VERAPREP Concentrate TSH protocol to produce a 1.0 mL Enriched Sample for subsequent TSH ELISA testing:
1. Dilute 80 μL of a 10 μIU/mL TSH standard to 0.0195 μIU/mL in 41.0 mL PBS, and save 1.0 mL as a Baseline Sample (prior to enrichment)
2. Dilute 80 μL of a 10 μIU/mL TSH standard to 0.80 μIU/mL in 1.0 mL VERAPREP Cleave as Control
3. Add 40 mL of 0.0195 μIU/mL TSH in PBS to a 50 mL Falcon Tube
4. Add VERAPREP Concentrate TSH, mix
5. Incubate 60 min at Room Temperature with mixing
6. Magnetically separate VERAPREP Concentrate TSH for 60 min using the Dexter LifeSep® 50SX
7. Decant and discard the 40 mL PBS into waste
8. Add 4.0 mL PBS Wash Buffer, mix
9. Magnetically separate VERAPREP Concentrate TSH in 4 mL PBS Wash Buffer for 30 min using the Dexter LifeSep® 50SX.
10. Decant and discard the 4 mL PBS into waste
11. Add 1 mL PBS Wash Buffer, mix
12. Transfer 1 mL VERAPREP Concentrate TSH to a 1.75 mL conical bottom snap-cap vial
13. Magnetically separate VERAPREP Concentrate TSH in 1 mL PBS Wash Buffer for 10 min using the Dexter LifeSep® 1.5S.
14. Aspirate and discard the 1 mL PBS into waste
15. Add 1 mL VERAPREP Cleave, mix
16. Magnetically separate VERAPREP Concentrate TSH in 1 mL VERAPREP Cleave for 10 min using the Dexter LifeSep® 1.5S.
17. Aspirate and save 1 mL supernatant (Enriched Sample), and test the Control, Baseline Sample and Enriched Sample.

0.08 mL TSH antigen (10 μIU/mL ELISA calibrator) was also diluted to 0.800 μIU/mL in 1 mL in VERAPREP Cleave buffer as the Control. The Baseline Sample, Enriched Sample, and Control were tested by the DRG TSH Ultrasensitive ELISA, and TSH % Recovery of the Enriched Sample was calculated as [Enriched Sample result]/[Control result]×100%. As expected, the diluted TSH Baseline Sample was undetectable by the Ultrasensitive ELISA and read 0.00 μIU/mL. Using only 0.80 mg reagent, VERAPREP Concentrate TSH successfully enriched the diluted TSH from undetectable to 0.73 μIU/mL (Table 1). As compared to the Control this was a 98.6% recovery, but there may have been a matrix effect of the VERAPREP Cleave buffer in the TSH ELISA that suppressed assay signal (Table 2).

TABLE 1

| | Theorectical TSH Result | Oberved TSH Result |
|---|---|---|
| Sample | (μIU/mL) | (μIU/mL) |
| Control | 0.8000 | 0.74 |
| Baseline Sample | 0.0195 | 0.00 |
| Enriched Sample | 0.7800 | 0.73 |

TABLE 2
% TSH Recovery

([Enriched Sample]/[Control]) × 100%
([0.73 μIU/mL]/[0.74 μIU/mL) × 100% = 98.6%

In the second study, VERAPREP Concentrate PTH reagent was prepared by coating 550 nm VERAPREP Biotin with biotinylated anti-PTH capture antibody. 0.021 mL PTH antigen (971 pg/mL ELISA calibrator) was diluted to 0.497 pg/mL in 41 mL PBS buffer below the Functional Sensitivity (<1.56 pg/mL) of the DRG PTH (Parathyroid) Intact ELISA (Part No. EIA-3645, Lot No. 2896), and 1 mL was saved as the Baseline Sample (prior to enrichment). The 40 mL sample was processed using the VERAPREP Concentrate PTH protocol to produce a 1.0 mL Enriched Sample for subsequent PTH ELISA testing:
1. Dilute 21 μL of a 971 pg/mL PTH standard to 0.497 pg/mL in 41.0 mL PBS, and save 1.0 mL as a Baseline Sample (prior to enrichment)
   2. Dilute 21 μL of a 971 pg/mL PTH standard to 20.4 pg/mL in 1.0 mL VERAPREP Cleave as Control
   3. Add 40 mL of 0.497 pg/mL PTH in PBS to a 50 mL Falcon Tube
   4. Add VERAPREP Concentrate PTH, mix
   5. Incubate 30 min at Room Temperature with mixing
   6. Magnetically separate VERAPREP Concentrate PTH for 15 min using the Dexter LifeSep® 50SX
   7. Decant and discard the 40 mL PBS into waste
   8. Add 4.0 mL PBS Wash Buffer, mix
   9. Magnetically separate VERAPREP Concentrate PTH in 4 mL PBS Wash Buffer for 10 min using the Dexter LifeSep® 50SX.
10. Decant and discard the 4 mL PBS into waste
11. Add 1 mL PBS Wash Buffer, mix
12. Transfer 1 mL VERAPREP Concentrate PTH to a 1.75 mL conical bottom snap-cap vial
13. Magnetically separate VERAPREP Concentrate PTH in 1 mL PBS Wash Buffer for 10 min using the Dexter LifeSep® 1.5S.
14. Aspirate and discard the 1 mL PBS into waste
15. Add 1 mL VERAPREP Cleave, mix
16. Magnetically separate VERAPREP Concentrate PTH in 1 mL VERAPREP Cleave for 10 min using the Dexter LifeSep® 1.5S.
17. Aspirate and save 1 mL supernatant (Enriched Sample), and test the Control, Baseline Sample and Enriched Sample.

0.021 mL PTH antigen (971 pg/mL ELISA calibrator) was also diluted to 20.4 pg/mL in 1 mL in VERAPREP Cleave buffer as the Control. The Baseline Sample, Enriched Sample, and Control were tested by the DRG PTH (Parathyroid) Intact ELISA, and PTH % Recovery of the Enriched Sample was calculated as [Enriched Sample result]/[Control result]×100%. The diluted PTH Baseline Sample read 13.5 pg/mL due to a matrix effect of the VERAPREP Cleave buffer in the ELISA assay. This matrix effect resulted in enhanced assay signal. Using only 0.80 mg reagent, VERAPREP Concentrate PTH successfully enriched the diluted PTH to 42.3 pg/mL (Table 3). As compared to the Control this was a 109% recovery (Table 4).

TABLE 3

| | Theorectical PTH Result | Oberved PTH Result |
|---|---|---|
| Sample | (pg/mL) | (pg/mL)) |
| Control | 20.4 | 38.8 |
| Baseline Sample | 0.497 | 13.5 |
| Enriched Sample | 20.0 | 42.3 |

TABLE 4
% PTH Recovery

([Enriched Sample]/[Control]) × 100%
([42.3 pg/mL]/[38.8 pg/mL) × 100% = 109%

### Example 4: Low Abundance Biomarker Enrichment from Urine for Subsequent Mass Spectrometry (LC-MS/MS or MALDI-MS) Analysis

The following describes a Mass Spectrometry sample pre-treatment protocol to enrich a low abundance biomarker and spiked internal standard (ISTD) from a large volume urine sample using superparamagnetic nanoparticles coated with a capture moiety specific for the biomarker. The exact same protocol could also use a plurality of different superparamagnetic nanoparticles populations mixed together or pooled together, where each population is coated with a different capture moiety, in order to multiplex and enrich more than 1 biomarker and corresponding spiked ISTD from the same sample. The enrichment and characterization of 2 or more biomarkers facilitates the use of an algorithm for the clinical diagnosis and/or prognosis of disease that is not possible with the characterization of a single biomarker. For example, for the diagnosis of obstructive sleep apnea (OSA) from urine, the VERAPREP Concentrate reagent could comprise 4 different antibodies to capture and enrich kallikrein-1, uromodulin, urocortin-3 and orosomucoid-1, or 7 different antibodies to capture and enrich kallikrein-1, uromodulin, urocortin-3 and orosomucoid-1, IL-6, IL-10 and high sensitivity C-reactive protein:
1. Collect patient urine (use standard urine collection protocol such as a urine collection cup)
2. Mix urine collection sample
3. Add 40 mL urine to the 50 mL Falcon Tube
4. Add Deuterated Internal Standard for the biomarker to be enriched, mix
5. Add VERAPREP Condition, mix
6. Add VERAPREP Concentrate, mix
7. Incubate: biomarker+deuterated internal standard capture by VERAPREP Concentrate
8. Magnetically separate VERAPREP Concentrate in the 40 mL urine using the Dexter LifeSep® 50SX
9. Aspirate and discard the urine into waste
10. Add 4 mL PBS Wash Buffer, mix
11. Magnetically separate VERAPREP Concentrate in 4 mL PBS Wash Buffer using the Dexter LifeSep® 50SX.
12. Aspirate and discard the urine into waste
13. Repeat Step 12 two more times (2×)
14. Add 1 mL VERAPREP Cleave, mix (Mass Spectrometry compatible buffer)
15. Magnetically separate VERAPREP Concentrate in 1 mL VERAPREP Cleave using the Dexter LifeSep® 1.5S.
16. Aspirate and Test the 1 mL supernatant sample by LC-MS
17. Final biomarker concentration determined based on: 1) 40 mL urine sample size, 2) LC-MS quantitation of the biomarker, and 3) adjust reported biomarker value based on Deuterated Internal Standard recovery

The selective release or cleavage of the captured and enriched biomarker or biomarkers can be accomplished with a change in pH (acidic pH such as glycine pH 2.5 elution followed by neutralization, or alkaline pH 10.0 or greater), using a cleavable linker such as a disulfide bond cleaved with a reducing agent such as TCEP or DTT, or by using competitive elution such a molar excess of D-biotin with monomeric avidin or molar excess of sugar with Concanavalin A that compete for the binding sites on Concanavalin A.

### Example 5: Sample Pre-Treatment to Deplete Sample Interferences Prior to Low Abundance Biomarker Enrichment

The following describes a sample pre-treatment protocol to deplete sample interferences prior to enriching a low abundance biomarker. In this protocol, a sample is pre-treated with a reagent A which comprises a microparticle that is exactly the same as the microparticle in reagent B, except the capture moiety on reagent A microparticles does not have specificity for the biomarker to be enriched. After sample pre-treatment to deplete sample interferences the reagent A beads is magnetically, physically or chemically removed from the sample. Next, the interference depleted or free sample is added or mixed with reagent B to capture and enrich the biomarker in the absence of interference.

This method is automated on a liquid handling system such as Hamilton or Tecan using magnets on the deck to capture the magnetic, paramagnetic or superparamagnetic microparticles, such as 96-well and 384-well plate magnets:
1. Sample rack is loaded on system;
2. Sample is aspirated and dispensed into Reaction Vessel A such as a tube, 96-well plate well, or 384-well plate well;
3. Mixed Reagent A (magnetic microparticles) is aspirated and dispensed into Reaction Vessel A, mixed and incubated to capture and deplete sample specific interference;
4. Reaction Vessel A is placed in a magnet position (e.g., a single magnet for a tube, or 96-well or 384-well magnet separator) to separate Reagent A;
5. Pre-treated Sample is aspirated and dispensed into Reaction Vessel B such as a tube, 96-well plate well, or 384-well plate well;
6. Mixed Reagent B is added to Reaction Vessel B, mixed and incubated to capture biomarker or enrich biomarker in the absence of interference (depleted and removed by Reagent A sample pre-treatment);
7. Reaction Vessel B is placed in a magnet position (e.g., a single magnet for a tube, or 96-well or 384-well magnet separator) to separate Reagent A;
8. The Sample supernatant and matrix is aspirated/removed and discarded to wash Reagent B;
9. Reagent B is tested directly in a measurement system to measure Biomarker captured, or the Biomarker iscleaved, eluted or selectively released from Reagent B, or the capture moiety-biomarker complex is cleaved, eluted or selectively released from Reagent B, or the (pre-labeled capture moiety)-biomarker complex cis cleaved, eluted or selectively released from Reagent B for measurement in a test system.

### Abbreviations

ABEI N-(4-aminobutyl)-N-ethylisoluminol
ALP Alkaline phosphatase
BSA Bovine serum albumin
Fab Fragment antibody-binding
Fc Fragment, crystallizable
HAAA Human anti-animal antibody
HAMA Human anti-mouse antibody
HASA Human anti-sheep antibody
IFU Instructions for use
IgG Antibody or immunoglobulin
IgM Immunoglobulin M
HRP Horse radish peroxidase
LC-MS/MS Liquid chromatography tandem-mass spectrometry
LDT Laboratory developed test
Mab Monoclonal antibody
MASI Manufacture assay specific interference
MFG IVD Manufacturers
PMP Superparamagnetic microparticles
PBCT Primary blood collection tubes
RF Rheumatoid Factor
RLU Relative light units or assay response signal
RUO Research use only
SAv Streptavidin
STT Secondary transfer tubes
TAT Turnaround time
WF Work flow

### Definitions

As used herein, “sample” or “biological sample” refers to any human or animal serum, plasma (i.e. EDTA, lithium heparin, sodium citrate), blood, whole blood, processed blood, urine, saliva, stool (liquid and solid), semen or seminal fluid, amniotic fluid, cerebral spinal fluid, cells, tissues, biopsy material, DNA, RNA, or any fluid, dissolved solid, or processed solid material to be tested for diagnosis, prognosis, screening, risk assessment, risk stratification, and monitoring such as therapeutic drug monitoring. In some embodiments, the sample is a large volume sample. In some embodiments, the sample comprises a plurality of samples (e.g., more than one sample from the same or a different subject. In some embodiments, the sample comprises a biomarker present at low abundance in the sample.

In some embodiments, the sample is collected into in a primary blood collection tube (PBCT), secondary transfer tube (SST), 24-hour (24-hr) urine collection device, BD Vacutainer Barricor tubes, nanotainer, a saliva collection tube, blood spot filter paper, or any collection tube or device such as for stool and seminal fluid, a light green top or green top plasma separator tube (PST) containing sodium heparin, lithium heparin or ammonium heparin, a light blue top tube containing sodium citrate (i.e. 3.2% or 3.8%) or citrate, theophylline, adenosine, dipyridamole (CTAD), a red top tube for Serology or Immunohematology for the collection of serum in a glass (no additives) or plastic tube (contains clot activators), a red top tube for Chemistry for the collection of serum in a glass (no additives) or plastic tube (contains clot activators), a purple lavender top tube containing EDTA K2, EDTA K3, liquid EDTA solution (i.e. 8%), or EDTA K2/gel tubes for testing plasma in molecular diagnostics and viral load detection, a pink top tube for Blood Bank EDTA, a gray top tube containing potassium oxalate and sodium fluoride, sodium fluoride/EDTA, or sodium fluoride (no anticoagulant, will result in a serum sample), a yellow top tube containing ACD solution A or ACD solution B, a royal blue top (serum, no additive or sodium heparin), a white top tube, or any color or tube type, for any application or diagnostic test type, containing no additives or any additive or combinations thereof, for the collection of blood.

In some embodiments, the sample is a challenging sample type such as urine, 24-hour urine, saliva and stool, or where a biomarker of interest may be dilute or difficult to measure. For example, the biological sample can be a challenging because of the patient population (e.g., neonatal, pediatric, geriatric, pregnant, oncology, autoimmune disease). For example, some biomarkers are too dilute or at too low of concentration, e.g., in circulation, or in urine, to be reliably detected and accurately and precisely measured by existing POCT and central laboratory analyzers. In some embodiments, the challenging sample is cerebrospinal fluid (CSF).

As used herein, “collection device” can be a primary blood collection tube (PBCT), 24-hr urine collection device, a urine collection device, a saliva collection tube, a stool collection device, a seminal fluid collection device, a blood collection bag, or any sample collection tube or device, prior to the addition of the sample.

A PBCT and secondary transfer tube (SST) can be any commercially available standard or custom collection tube (with or without gel separators) from companies like Becton Dickinson (BD), Greiner, VWR, and Sigma Aldrich, a glass tube, a plastic tube, a light green top or green top plasma separator tube (PST) containing sodium heparin, lithium heparin or ammonium heparin, light blue top tube containing sodium citrate (i.e. 3.2% or 3.8%) or citrate, theophylline, adenosine, dipyridamole (CTAD), red top tube for Serology or Immunohematology for the collection of serum in a glass (no additives) or plastic tube (contains clot activators), a red top tube for Chemistry for the collection of serum in a glass (no additives) or plastic tube (contains clot activators), a purple lavender top tube containing EDTA K2, EDTA K3, liquid EDTA solution (i.e. 8%), or EDTA K2/gel tubes for testing plasma in molecular diagnostics and viral load detection, a pink top tube for Blood Bank EDTA, a gray top tube containing potassium oxalate and sodium fluoride, sodium fluoride/EDTA, or sodium fluoride (no anticoagulant, will result in a serum sample), a yellow top tube containing ACD solution A or ACD solution B, a royal blue top (serum, no additive or sodium heparin), a white top tube, or any color or tube type, for any application or diagnostic test type, containing no additives or any additive or combinations thereof, for the collection of blood.

As used herein, a “storage device” or “transfer device” refers to a device that receives the sample and/or other components received in a collection device. The storage or transfer device can be a plastic or glass tube, vial, bottle, beaker, flask, bag, can, microtiter plate, ELISA plate, 96-well plate, 384-well plate 1536 well plate, cuvette, reaction module, reservoir, or any container suitable to hold, store or process a liquid sample.

As referred to herein, a “diagnostic test” includes, but is not limited to any antibody-based diagnostic test, non-antibody based diagnostic test, a sample pre-treatment method or device for subsequent analysis by chromatographic, spectrophotometric, and mass spectrometry methods (i.e. HPLC, MS, LCMS, LC-MS/MS) such as immunoextraction (IE) and solid phase extraction (SPE), radioimmunoassay (RIA), enzyme-linked immunoassay (ELISA), chemiluminescence immunoassay (CLIA), molecular diagnostics, lateral flow (LF), point-of-care (PoC), direct to consumer (DTC), CLIA and CLIA waived tests and devices, Research Use Only (RUO) test, In Vitro Diagnostics (IVD) test, Laboratory Developed Test (LDT), companion diagnostic, and any test for diagnosis, prognosis, screening, risk assessment, risk stratification, and monitoring such as therapeutic drug monitoring. In some embodiments, the diagnostic test comprises short turn-around time (STAT) diagnostic tests, ambulatory tests, lateral flow tests, point of care (PoC) tests, molecular diagnostic tests, HPLC, MS, LCMS, LC-MS/MS, radioimmunoassay (RIA), enzyme-linked immunoassay (ELISA), chemiluminescence immunoassay (CLIA), CLIA and CLIA waived tests, and any diagnostic test used for the diagnosis, prognosis, screening, risk assessment, risk stratification, treatment monitoring, and therapeutic drug monitoring.

What is claimed is:

1. A method for removing biomarkers from a biological sample, the method comprising:
a) combining the sample with a plurality of particles to provide a mixture, wherein each particle independently comprises a type or species of capture moiety and wherein the plurality of particles comprises at least a first particle comprising a first type or species of capture moiety and a second particle comprising a second type or species of capture moiety;
b) mixing the mixture to provide one or more particle complexes to the biomarkers; and
c) removing or isolating the particle complexes to provide an depleted solution;
thereby removing biomarkers from the biological sample.

2. A method for isolating biomarkers from a biological sample, the method comprising:
a) combining the sample with a plurality of particles to provide a mixture, wherein each particle independently comprises a type or species of capture moiety and wherein the plurality of particles comprises at least a first particle comprising a first type or species of capture moiety and a second particle comprising a second type or species of capture moiety;
b) the biomarkers; and
c) removing or isolating the particle complexes to provide a depleted solution and an enriched isolate;
thereby isolating biomarkers from the biological sample.

3. The method of claim 1 or 2, wherein a conditioning agent is added to the biological sample prior to combining the sample with the plurality of particles.

4. The method of claim 3, wherein the conditioning agent is a pH adjustment agent, a molarity adjustment agent, an interference blocking agent, or a liberation or release agent.

5. The method of claim 1 or 2, wherein the method is performed prior to performing a diagnostic test on the biological sample.

6. The method of claim 1 or 2, wherein the plurality of particles comprises a plurality of capture moieties (e.g., a plurality of particles each independently covalently or non-covalently bonded to a plurality of capture moieties).

7-8. (canceled)

9. The method of claim 1, wherein the plurality of particles comprises from a third to a tenth particle each comprising a third to a tenth type or species of capture moiety.

10-16. (canceled)

17. The method of claim 9, comprising removing or isolating a first, second, third, fourth, fifth, sixth, seventh, eighth, ninth, or tenth biomarker(s) from the biological sample.

18. The method of claim 1, wherein the method further comprises adding to the mixture a cleavage reagent or releasing agent to provide an enriched isolate.

19. The method of claim 1, further comprising performing a diagnostic test on the biomarker(s) (e.g., after the method of removing or method of isolating described herein).

20. The method of claim 19, wherein the diagnostic test detects the presence or absence of two or more biomarkers concurrently.

21. The method of claim 9, wherein the first particle is different in size, shape, chemistry, color, or other characteristic than at least one of the second though tenth particle.

22-29. (canceled)

30. The method of claim 21, wherein the characteristic is selectivity, affinity, or avidity to a biomarker.

31. The method of claim 21, wherein the size is 50-1000 nm (e.g., 100-500 nm, 200-600 nm) in diameter.

32. The method of claim 21, wherein the size is 1-3 micron in diameter.

33. The method of claim 1, wherein a first population of particles is present at a greater concentration than a second population of particles.

34. The method of claim 33, wherein the ratio of a first particle to a second particle is 1:1, 1:2, 1:3, 1:4, 1:5, 1:6, 1:7, 1:8, 1:9, 1:10.

35. The method of claim 1, wherein the first particle is present at a first concentration that is different than the concentration of at least one of the second though tenth particle.

36-44. (canceled)

45. The method of claim 1, wherein the first particle is a control particle (e.g., particle comprising a label or indicator (e.g., a label or indicator of known quantity, abundance).

46. The method of claim 45, wherein the label or indicator provides measurement of concentration or volume of the sample.

47. The method of claim 45, wherein the label or indicator provides a measurement of yield or particle recovery.

48. The method of claim 45, wherein the label or indicator provides indication of lot number or batch number of the sample.

49. The method of claim 1, wherein the biomarker(s) is biotin, HAMA, RF, Heterophilic, or anti-SAv.

50. The method of claim 1, wherein the biomarker(s) is an indicator of bacterial infection.

51. The method of claim 1, wherein the biomarker is a capture moiety for a bacterium.

52. The method of claim 1, wherein removing or isolating the particle complexes comprises cleaving, eluting, or selectively releasing a capture moiety-biomarker complex.

53. The method of claim 52, wherein the capture moiety comprises a signal detection molecule for measurement in a test system.

54. The method of claim 1, wherein prior to the combining step a), the sample is pre-treated to remove or deplete an interference, comprising: (i) combining the sample with a particle comprising a capture moiety lacking specificity for the biomarkers to provide a mixture; (ii) mixing the mixture to provide particle complexes to the interference; and (iii) removing or eliminating the particle complexes to provide a depleted solution.

55. A kit comprising a plurality of particles, magnet, tube and an instruction booklet.

56-60. (canceled)
